(19) United States
(12) Patent Application Publication
(10) Pub. No.: US 2003/0166001 A1
(43) Pub. Date: Sep. 4, 2003

(54) Toll-like receptor 3 signaling agonists and antagonists

(76) Inventor: Grayson B. Lipford, Dusseldorf (DE)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 10/265,072

(22) Filed: Oct. 5, 2002

Related U.S. Application Data
(60) Provisional application No. 60/327,520, filed on Oct. 5, 2001.

(51) Int. Cl. G01N 33/53; G06F 19/00; G01N 33/48; G01N 33/50

(52) U.S. Cl. 435/7.1; 702/19

(57) ABSTRACT

Compositions and methods are provided to identify, characterize, and optimize immunostimulatory compounds, their agonists and antagonists, working through TLR3.

### RELATED APPLICATION

This application claims benefit of U.S. provisional patent application Serial No. 60/327,520, filed Oct. 5, 2001.

### FIELD OF THE INVENTION

The invention pertains to signal transduction by Toll-like receptor 3 (TLR3), which is believed to be involved in innate immunity. More specifically, the invention pertains to screening methods useful for the identification and characterization of TLR3 ligands, TLR3 signaling agonists, and TLR3 signaling antagonists.

### BACKGROUND OF THE INVENTION

Toll-like receptors (TLRs) are a family of at least ten highly conserved receptor proteins (TLR1-TLR10) which recognize pathogen-associated molecular patterns (PAMPs) and act as key elements in innate immunity. As members of the pro-inflammatory interleukin-1 receptor (IL-1R) family, TLRs share homologies in their cytoplasmic domains called Toll/IL-1R homology (TIR) domains. PCT published applications PCT/US98/08979 and PCT/USO1/16766. Intracellular signaling mechanisms mediated by TIRs appear generally similar, with MyD88 (Wesche H et al. (1997) Immunity 7:837-47; Medzhitov R et al. (1998) Mol Cell 2:253-8; Adachi O et al. (1998) Immunity 9:143-50; Kawai T et al. (1999) Immunity 11:115-22) and tumor necrosis factor receptor-associated factor 6 (TRAF6; Cao Z et al. (1996) Nature 383:443-6; Lomaga M A et al. (1999) Genes Dev 13:1015-24) believed to have critical roles. Signal transduction between MyD88 and TRAF6 is known to involve members of the serine-threonine kinase IL-1 receptor-associated kinase (IRAK) family, including at least IRAK-1 and IRAK-2. Muzio M et al. (1997) Science 278:1612-5.

Ligands for many but not all of the TLRs have been described. For instance, it has been reported that TLR2 signals in response to peptidoglycan and lipopeptides. Yoshimura A et al. (1999) J Immunol 163:1-5; Brightbill H D et al. (1999) Science 285:732-6; Aliprantis A O et al. (1999) Science 285:736-9; Takeuchi O et al. (1999) Immunity 11:443-51; Underhill D M et al. (1999) Nature 401:811-5. TLR4 has been reported to signal in response to lipopolysaccharide (LPS). Hoshino K et al. (1999) J Immunol 162:3749-52; Poltorak A et al. (1998) Science 282:2085-8; Medzhitov R et al. (1997) Nature 388:394-7. Bacterial flagellin has been reported to be a natural ligand for TLR5. Hayashi F et al. (2001) Nature 410:1099-1103. TLR6, in conjunction with with TLR2, has been reported to signal in response to proteoglycan. Ozinsky A et al. (2000) PNAS USA 97:13766-71; Takeuchi O et al. (2001) Int Immunol 13:933-40. Recently it was recently reported that TLR9 is a receptor for CpG DNA. Hemmi H et al. (2000) Nature 408:740-5.

### SUMMARY OF THE INVENTION

The invention provides screening methods and compositions useful for the identification and characterization of compounds which themselves signal through Toll-like receptor 3 (TLR3) or which influence signaling through TLR3. Compounds which themselves signal through TLR3 are presumptively immunostimulatory. Compounds which influence signaling through TLR3 include both agonists and antagonists of TLR3 signaling activity. The methods provided by the invention are adaptable to high throughput screening, thus accelerating the identification and characterization of previously unknown inducers, agonists, and antagonists of TLR3 signaling activity.

The methods of the invention rely at least in part on the ability to assess TLR3 signaling activity. It has surprisingly been discovered according to the present invention that reporter constructs having reporter genes under control of certain promoter response elements sensitive to TLR3 signaling activity are useful in the screening assays of the invention. For example it has been surprisingly discovered according to the present invention that a reporter gene under control of interferon-specific response element (ISRE) is sensitive to TLR3 signaling activity.

It has also surprisingly been discovered according to the present invention that screening assays for TLR ligands and other assays involving TLR signaling activity can benefit from optimization for at least one of the variables of (a) concentration of test and/or reference compound, (b) kinetics of the assay, and (c) selection of reporter. Interpretation of assay data can be influenced by each of these variables.

In one aspect the invention provides a screening method for identifying an immunostimulatory compound. The method according to this aspect of the invention involves the steps of (a) contacting a functional TLR3 with a test compound under conditions which, in absence of the test compound, permit a negative control response mediated by a TLR3 signal transduction pathway; (b) detecting a test response mediated by the TLR3 signal transduction pathway; and (c) determining the test compound is an immunostimulatory compound when the test response exceeds the negative control response. In this and in all aspects of the invention, in one embodiment the screening method is performed on a plurality of test compounds. A test compound according to this and all aspects of the invention is in one embodiment a member of a library of compounds, preferably a combinatorial library of compounds. Also in this and in all aspects of the invention, a test compound is preferably a small molecule, a nucleic acid, a polypeptide, an oligopeptide, or a lipid. In more preferred embodiments, the test compound is a small molecule or a nucleic acid. In one embodiment a test compound that is a nucleic acid is a CpG nucleic acid.

In another aspect the invention provides a screening method for identifying an immunostimulatory compound. The method according to this aspect of the invention involves the steps of (a) contacting a functional TLR3 with a test compound under conditions which, in presence of a reference immunostimulatory compound, permit a reference response mediated by a TLR3 signal transduction pathway; (b) detecting a test response mediated by the TLR3 signal transduction pathway; and (c) determining the test compound is an immunostimulatory compound when the test response equals or exceeds the reference response. In this and other aspects of the invention, a reference immunostimulatory compound is preferably a small molecule, a nucleic acid, a polypeptide, an oligopeptide, or a lipid. In one embodiment the reference immunostimulatory compound is a CpG nucleic acid.

In a further aspect the invention provides a screening method for identifying a compound that modulates TLR3 signaling activity. The method according to this aspect of the invention involves the steps of (a) contacting a functional TLR3 with a test compound and a reference immunostimulatory compound under conditions which, in presence of the reference immunostimulatory compound alone, permit a reference response mediated by a TLR3 signal transduction pathway; (b) detecting a test-reference response mediated by the TLR3 signal transduction pathway; (c) determining the test compound is an agonist of TLR3 signaling activity when the test-reference response exceeds the reference response; and (d) determining the test compound is an antagonist of TLR3 signaling activity when the reference response exceeds the test-reference response.

In yet another aspect the invention provides a screening method for identifying species specificity of an immunostimulatory compound. The method according to this aspect of the invention involves the steps of (a) measuring a first species-specific response mediated by a TLR3 signal transduction pathway when a functional TLR3 of a first species is contacted with a test compound; (b) measuring a second species-specific response mediated by the TLR3 signal transduction pathway when a functional TLR3 of a second species is contacted with the test compound; and (c) comparing the first species-specific response with the second species-specific response. In a preferred embodiment the functional TLR3 of the first species is a human TLR3. In one preferred embodiment the functional TLR3 of the first species is a human TLR3 and the functional TLR3 of the second species is a mouse TLR3.

In preferred embodiments of the foregoing aspects of the invention, the response mediated by the TLR3 signal transduction pathway is measured quantitatively.

Also in preferred embodiments of the foregoing aspects of the invention, the functional TLR3 is expressed in a cell. For example, in one embodiment the cell is an isolated mammalian cell that naturally expresses the functional TLR3. Alternatively, in another embodiment the cell is an isolated mammalian cell that does not naturally express the functional TLR3, wherein the cell has an expression vector for TLR3. For example, in one preferred embodiment the cell is a human 293 fibroblast. In other embodiments, the functional TLR3 is part of a cell-free system.

Particularly useful in embodiments of the invention involving cells which express functional TLR3 are cells which include a reporter construct sensitive to TLR3 signaling. In one embodiment the cell includes an expression vector having an isolated nucleic acid which encodes a reporter construct selected from the group of nuclear factor-kappa B-luciferase (NF-κB-luc), IFN-specific response element-luciferase (ISRE-luc), interleukin-6-luciferase (IL-6-luc), interleukin 8-luciferase (IL-8-luc), interleukin 12 p40 subunit-luciferase (IL-12 p40-luc), interleukin 12 p40 subunit-beta galactosidase (IL-12 p40-β-Gal), activator protein 1-luciferase (AP1-luc), interferon alpha-luciferase (IFN-α-luc), interferon beta-luciferase (IFN-β-luc), RANTES-luciferase (RANTES-luc), tumor necrosis factor-luciferase (TNF-luc), IP-10-luciferase (IP-10-luc), and interferon-inducible T cell alpha chemoattractant-luciferase (I-TAC-luc). In a preferred embodiment the reporter construct is ISRE-luc.

In one embodiment according to each of the foregoing aspects of the invention, the functional TLR3 is part of a complex with a non-TLR protein selected from the group consisting of MyD88, IL-1 receptor associated kinase 1-3 (IRAK1, IRAK2, IRAK3), tumor necrosis factor receptor-associated factor 1-6 (TRAF1-TRAF6), IκB, NF-κB, MyD88-adapter-like (Mal), Toll-interleukin 1 receptor (TIR) domain-containing adapter protein (TIRAP), Tollip, Rac, and functional homologues and derivatives thereof. In a related embodiment functional TLR3 is part of a complex with a non-TLR protein listed above, excluding MyD88.

Also according to each of the foregoing aspects of the invention, in one embodiment the response mediated by a TLR3 signal transduction pathway is induction of a reporter gene under control of a promoter response element selected from the group consisting of ISRE, IL-6, IL-8, IL-12 p40, IFN-α, IFN-β, IFN-ω, RANTES, TNF, IP-10, and I-TAC. For example, in a preferred embodiment the reporter gene under control of a promoter response element is selected from the group consisting of ISRE-luc, IL-6-luc, IL-8-luc, IL-12 p40-luc, IL-12 p40-β-Gal, IFN-α-luc, IFN-β-luc, RANTES-luc, TNF-luc, IP-10-luc, and I-TAC-luc. In one preferred embodiment the reporter gene under control of a promoter response element is ISRE-luc. In yet another preferred embodiment the reporter gene is selected from the group consisting of IFN-α1-luc and IFN-α4-luc.

In yet another embodiment according to each of the foregoing aspects of the invention, the response mediated by a TLR3 signal transduction pathway is selected from the group consisting of (a) induction of a reporter gene under control of a minimal promoter responsive to a transcription factor selected from the group consisting of AP1, NF-κB, ATF2, IRF3, and IRF7; (b) secretion of a chemokine; and (c) secretion of a cytokine. For example, in one preferred embodiment the response mediated by a TLR3 signal transduction pathway is induction of a reporter gene selected from the group consisting of AP1-luc and NF-κB-luc. In another preferred embodiment the response mediated by a TLR3 signal transduction pathway is secretion of a type 1 IFN. In yet another preferred embodiment the response mediated by a TLR3 signal transduction pathway is secretion of a chemokine selected from the group consisting of CCL5 (RANTES), CXCL9 (Mig), CXCL10 (IP-10), and CXCL11 (1-TAC).

The sensitivity and interpretation of the screening methods of the present invention can be optimized. Such optimization involves proper selection of any one or combination of (a) concentration of test and/or reference compound, (b) kinetics of the assay, and (c) reporter. Thus, further according to each of the first three aspects of the invention, in one embodiment the contacting a functional TLR3 with a test compound further entails, for each test compound, contacting with the test compound at each of a plurality of concentrations. For example, each test compound may be evaluated at various concentrations which differ by log increments. Also according to each of the foregoing aspects of the invention, in one embodiment the detecting is performed 4-12 hours, preferably 6-8 hours, following the contacting. Similarly, in yet another embodiment according to each of the foregoing aspects of the invention, the detecting is performed 16-24 hours following the contacting. Detecting performed 4-12 hours, preferably 6-8 hours, following the contacting is believed to be more sensitive to affinity of interaction than is detecting at later times. Detecting performed 16-24 hours or later following the contacting is believed to be more sensitive to stability and duration of receptor/ligand interaction. Furthermore, because certain reporter constructs are more sensitive to certain TLRs than others, proper matching of reporter to TLR assay is important to increase signal-to-noise ratio in the readout of a particular assay.

### BRIEF DESCRIPTION OF THE FIGURES

This application includes examples which refer to figures or other drawings. It is to be understood that the referenced figures are illustrative only and are not essential to the enablement of the claimed invention.

FIG. 1 is two paired bar graphs showing (A) the induction of NF-κB and (B) the amount of IL-8 produced by 293 fibroblast cells transfected with human TLR9 in response to exposure to various stimuli, including CpG-ODN, GpC-ODN, LPS, and medium.

FIG. 2 is a bar graph showing the induction of NF-κB produced by 293 fibroblast cells transfected with murine TLR9 in response to exposure to various stimuli, including CpG-ODN, methylated CpG-ODN (Me-CpG-ODN), GpC-ODN, LPS, and medium.

FIG. 3 is a series of gel images depicting the results of reverse transcriptase-polymerase chain reaction (RT-PCR) assays for murine TLR9 (mTLR9), human TLR9 (hTLR9), and glyceraldehyde-3-phosphate dehydrogenase (GAPDH) in untransfected control 293 cells, 293 cells transfected with mTLR9 (293-mTLR9), and 293 cells transfected with hTLR9 (293-hTLR9).

FIG. 4 is a graph showing the degree of induction of NF-κB-luc by various stimuli in stably transfected 293-hTLR9 cells.

FIG. 5 is a graph showing the degree of induction of NF-κB-luc by various stimuli in stably transfected 293-mTLR9 cells.

FIG. 6 is a graph showing fold induction of response as a function of concentration for a series of four related immunostimulatory nucleic acids contacted with human 293 fibroblast cells stably transfected with murine TLR9 and NF-κB-luc. Concentrations listed correspond to EC50 for each ligand.

FIG. 7 is a graph showing kinetics of EC50 determinations for a series of five immunostimulatory nucleic acids contacted with human 293 fibroblast cells stably transfected with murine TLR9 and NF-κB-luc.

FIG. 8 is a graph showing kinetics of EC50 determinations for the same series of five immunostimulatory nucleic acids as in FIG. 7 contacted with human 293 fibroblast cells stably transfected with human TLR9 and NF-κB-luc.

FIG. 9 is a graph showing kinetics of maximal activity (fold induction of response) for the same series of five immunostimulatory nucleic acids as in FIG. 7 contacted with human 293 fibroblast cells stably transfected with murine TLR9 and NF-κB-luc.

FIG. 10 is a graph showing kinetics of maximal activity (fold induction of response) for the same series of five immunostimulatory nucleic acids as in FIG. 7 contacted with human 293 fibroblast cells stably transfected with human TLR9 and NF-κB-luc.

FIG. 11 is a bar graph showing fold induction of response as measured using various luciferase reporter constructs (NF-κB-luc, IP-10-luc, RANTES-luc, ISRE-luc, and IL-8-luc) in combination with TLR7, TLR8, and TLR9, each TLR contacted with a specific reference TLR ligand.

### DETAILED DESCRIPTION OF THE INVENTION

The invention in certain aspects provides screening methods useful for the identification, characterization, and optimization of immunostimulatory compounds, including but not limited to immunostimulatory nucleic acids and immunostimulatory small molecules, as well as assays for the identification and optimization of agonists and antagonists of TLR3 signaling. The methods according to the invention include both cell-based and cell-free assays. In certain preferred embodiments the screening methods are performed in a high throughput manner. The methods can be used to screen libraries of compounds for their ability to modulate immune activation that involves TLR3 signaling.

In one aspect the invention provides a screening method for identifying an immunostimulatory compound. The method according to this aspect of the invention involves the steps of (a) contacting a functional TLR3 with a test compound under conditions which, in absence of the test compound, permit a negative control response mediated by a TLR3 signal transduction pathway; (b) detecting a test response mediated by the TLR3 signal transduction pathway; and (c) determining the test compound is an immunostimulatory compound when the test response exceeds the negative control response. In a second aspect the invention provides a screening method for identifying an immunostimulatory compound. The method according to this aspect of the invention involves the steps of (a) contacting a functional TLR3 with a test compound under conditions which, in presence of a reference immunostimulatory compound, permit a reference response mediated by a TLR3 signal transduction pathway; (b) detecting a test response mediated by the TLR3 signal transduction pathway; and (c) determining the test compound is an immunostimulatory compound when the test response equals or exceeds the reference response. It will be appreciated that these two aspects of the invention differ in that one involves comparison of the test compound against a negative control and the other involves comparison of the test compound against a positive control.

For these and other aspects of the invention, the TLR3 is preferably a mammalian TLR3, such as human TLR3 or mouse TLR3. Nucleotide and amino acid sequences for human TLR3 and murine TLR3 have previously been described. The nucleotide sequence for human TLR3 cDNA can be found as GenBank accession no. NM_{—}003265 (SEQ ID NO:1), and the deduced amino acid sequence for human TLR3, encompassing 904 amino acids, can be found as GenBank accession nos NP_{—}003256 (SEQ ID NO:2). The nucleotide sequence for murine TLR3 cDNA can be found as GenBank accession no. AF355152 (SEQ ID NO:3), and the deduced amino acid sequence for murine TLR3, encompassing 905 amino acids, can be found as GenBank accession no. AAK26117 (SEQ ID NO:4).

As used herein, a “functional TLR3” shall refer to a polypeptide, including a full length naturally occurring TLR3 polypeptide as described above, which specifically binds a TLR3 ligand and signals via a Toll/interleukin-1 receptor (TIR) domain. In addition to full length naturally occurring TLR3, a functional TLR3 thus also refers to allelic variants, fusion proteins, and truncated versions of the same, provided the polypeptide specifically binds a TLR3 ligand and signals via a TIR domain. In a preferred embodiment, the functional TLR3 includes a human TLR3 extracellular domain having an amino acid sequence provided by amino acids 38-707 according to SEQ ID NO:2. In another preferred embodiment, the functional TLR3 includes a murine TLR3 extracellular domain having an amino acid sequence provided by amino acids 39-708 according to SEQ ID NO:4. Preferably, the functional TLR3 signals through a TIR domain of TLR3.

In certain embodiments of this and other aspects of the invention, the functional TLR3 is expressed, either naturally or artifically, in a cell. In some embodiments, a cell expressing TLR3 for use in the methods of the invention expresses TLR3 and no other TLR. Alternatively, in some embodiments a cell expressing TLR3 for use in the methods of the invention expresses both TLR3 and at least one other TLR, e.g., TLR7, TLR8, or TLR9. In one embodiment the cell is an isolated mammalian cell that naturally expresses functional TLR3. Cells and tissues known to express TLR3 include dendritic cells (DCs), intraepithelial cells, and placenta. Muzio M et al. (2000) J Immunol 164:5998-6004; Cario E et al. (2000) Infect Immun 68:7010-7; Rock F L et al. (1998) Proc Natl Acad Sci USA 95:588-93. The term “isolated” as used herein, with reference to a cell or to a compound, means substantially free of or separated from components with which the cell or compound is normally associated in nature, e.g., other cells, nucleic acids, proteins, lipids, carbohydrates or in vivo systems to an extent practical and appropriate for its intended use.

In another embodiment the cell can be one that, as it occurs in nature, is not capable of expressing TLR3 but which is rendered capable of expressing TLR3 through the artificial introduction of an expression vector for TLR3. Examples of cell lines lacking TLR3 include, but are not limited to, human 293 fibroblasts (ATCC CRL-1573) and HEp-2 human epithelial cells (ATCC CCL-23). Examples of cell lines lacking TLR9 include, but are not limited to, human 293 fibroblasts (ATCC CRL-1573), MonoMac-6, THP-1, U937, CHO, and any TLR9 knock-out. Typically the cell, whether it is capable of expressing TLR3 naturally or artificially, preferably has all the necessary elements for signal transduction initiated through the the TLR3 receptor. For example, it is believed that TLR9 signaling requires the adapter protein MyD88 in an early step of signal transduction. In contrast, TLR3 appears not to require MyD88 but may require other factors further downstream, e.g., factors that induce mitogen-activated protein kinase (MAPK) and factors downstream of MAPK.

When indicated, introduction of a particular TLR into a cell or cell line is preferably accomplished by transient or stable transfection of the cell or cell line with a TLR-encoding nucleic acid sequence operatively linked to a gene expression sequence (as described herein). For example, a cell artificially induced to express TLR3 for use in the methods of the invention includes a cell that has been transiently or stably transfected with a TLR3 expression vector. Any suitable method of transient or stable transfection can be employed for this purpose.

An expression vector for TLR3 will include at least a nucleotide sequence coding for a functional TLR3 polypeptide, operably linked to a gene expression sequence which can direct the expression of the TLR3 nucleic acid within a eukaryotic or prokaryotic cell. A “gene expression sequence” is any regulatory nucleotide sequence, such as a promoter sequence or promoter-enhancer combination, which facilitates the efficient transcription and translation of the nucleic acid to which it is operably linked. With respect to TLR3 nucleic acid, the “gene expression sequence” is any regulatory nucleotide sequence, such as a promoter sequence or promoter-enhancer combination, which facilitates the efficient transcription and translation of the TLR3 nucleic acid to which it is operably linked. The gene expression sequence may, for example, be a mammalian or viral promoter, such as a constitutive or inducible promoter. Constitutive mammalian promoters include, but are not limited to, the promoters for the following genes: hypoxanthine phosphoribosyl transferase (HPRT), adenosine deaminase, pyruvate kinase, β-actin promoter, and other constitutive promoters. Exemplary viral promoters which function constitutively in eukaryotic cells include, for example, promoters from the simian virus (e.g., SV40), papillomavirus, adenovirus, human immunodeficiency virus (HIV), Rous sarcoma virus (RSV), cytomegalovirus (CMV), the long terminal repeats (LTR) of Moloney murine leukemia virus and other retroviruses, and the thymidine kinase (TK) promoter of herpes simplex virus. Other constitutive promoters are known to those of ordinary skill in the art. The promoters useful as gene expression sequences of the invention also include inducible promoters. Inducible promoters are expressed in the presence of an inducing agent. For example, the metallothionein (MT) promoter is induced to promote transcription and translation in the presence of certain metal ions. Other inducible promoters are known to those of ordinary skill in the art.

In general, the gene expression sequence shall include, as necessary, 5′ non-transcribing and 5′ non-translating sequences involved with the initiation of transcription and translation, respectively, such as a TATA box, capping sequence, CAAT sequence, and the like. Especially, such 5′ non-transcribing sequences will include a promoter region which includes a promoter sequence for transcriptional control of the operably joined TLR3 nucleic acid. The gene expression sequences optionally include enhancer sequences or upstream activator sequences as desired.

Generally a nucleic acid coding sequence and a gene expression sequence are said to be “operably linked” when they are covalently linked in such a way as to place the transcription and/or translation of the nucleic acid coding sequence under the influence or control of the gene expression sequence. Thus the TLR3 nucleic acid sequence and the gene expression sequence are said to be “operably linked” when they are covalently linked in such a way as to place the transcription and/or translation of the TLR3 coding sequence under the influence or control of the gene expression sequence. If it is desired that the TLR3 sequence be translated into a functional protein, two DNA sequences are said to be operably linked if induction of a promoter in the 5′ gene expression sequence results in the transcription of the TLR3 sequence and if the nature of the linkage between the two DNA sequences does not (1) result in the introduction of a frame-shift mutation, (2) interfere with the ability of the promoter region to direct the transcription of the TLR3 sequence, or (3) interfere with the ability of the corresponding RNA transcript to be translated into a protein. Thus, a gene expression sequence would be operably linked to a TLR3 nucleic acid sequence if the gene expression sequence were capable of effecting transcription of that TLR3 nucleic acid sequence such that the resulting transcript might be translated into the desired protein or polypeptide.

In certain embodiments a TLR expression vector is constructed so as to permit tandem expression of two distinct TLRs, e.g., both TLR3 and a second TLR. Such a tandem expression vector can be used when it is desired to express two TLRs using a single transformation or transfection. Alternatively, a TLR3 expression vector can be used in conjunction with a second expression vector constructed so as to permit expression of a second TLR.

The screening assays can have any of a number of possible readout systems based upon a TLR/IL-1R signal transduction pathway. In preferred embodiments, the readout for the screening assay is based on the use of native genes or, alternatively, transfected or otherwise artificially introduced reporter gene constructs which are responsive to the TLR/IL-1R signal transduction pathway involving MyD88, TRAF, p38, and/or ERK. Häcker H et al. (1999) EMBO J. 18:6973-82. These pathways activate kinases including KB kinase complex and c-Jun N-terminal kinases. Thus reporter genes and reporter gene constructs particularly useful for the assays include, e.g., a reporter gene operatively linked to a promoter sensitive to NF-κB. Examples of such promoters include, without limitation, those for NF-κB, IL-1β, IL-6, IL-8, IL-12 p40, CD80, CD86, and TNF-α. The reporter gene operatively linked to the TLR-sensitive promoter can include, without limitation, an enzyme (e.g., luciferase, alkaline phosphatase, β-galactosidase, chloramphenicol acetyltransferase (CAT), etc.), a bioluminescence marker (e.g., green-fluorescent protein (GFP, U.S. Pat. No. 5,491,084), etc.), a surface-expressed molecule (e.g., CD25), and a secreted molecule (e.g., IL-8, IL-12 p40, TNF-α). In certain preferred embodiments the reporter is selected from IL-8, TNF-α, NF-κB-luciferase (NF-κB-luc; Häcker H et al. (1999) EMBO J. 18:6973-82), IL-12 p40-luc (Murphy T L et al. (1995) Mol Cell Biol 15:5258-67), and TNF-luc (Häcker H et al. (1999) EMBO J. 18:6973-82). In assays relying on enzyme activity readout, substrate can be supplied as part of the assay, and detection can involve measurement of chemiluminescence, fluorescence, color development, incorporation of radioactive label, drug resistance, or other marker of enzyme activity. For assays relying on surface expression of a molecule, detection can be accomplished using flow cytometry (FACS) analysis or functional assays. Secreted molecules can be assayed using enzyme-linked immunosorbent assay (ELISA) or bioassays. These and other suitable readout systems are well known in the art and are commercially available.

Thus a cell expressing a functional TLR3 and useful for the methods of the invention has, in some embodiments, an expression vector comprising an isolated nucleic acid which encodes a reporter construct useful for detecting TLR signaling. The expression vector comprising an isolated nucleic acid which encodes a reporter construct useful for detecting TLR signaling can include a reporter gene under control of a minimal promoter responsive to a transcription factor believed by the applicant to be activated as a consequence of TLR3 signaling. Examples of such minimal promoters include, without limitation, promoters for the following genes: AP1, NF-κB, ATF2, IRF3, and IRF7. In other embodiments the expression vector comprising an isolated nucleic acid which encodes a reporter construct useful for detecting TLR signaling can include a gene under control of a promoter response element selected from IL-6, IL-8, IL-12 p40 subunit, a type 1 IFN, RANTES, TNF, IP-10, I-TAC, and ISRE. The promoter response element generally will be present in multiple copies, e.g., as tandem repeats. For example, an ISRE-luciferase reporter construct useful in the invention is available from Stratagene (catalog no. 219092) and includes a 5×ISRE tandem repeat joined to a TATA box upstream of a luciferase reporter gene. As discussed further elsewhere herein, the reporter itself can be any gene product suitable for detection by methods recognized in the art. Such methods for detection can include, for example, measurement of spontaneous or stimulated light emission, enzyme activity, expression of a soluble molecule, expression of a cell surface molecule, etc.

As mentioned above, the functional TLR3 is contacted with a test compound in order to identify an immunostimulatory compound. An immunostimulatory compound is a natural or synthetic compound that is capable of inducing an immune response when contacted with an immune cell. In the context of the methods of the invention, an immunostimulatory compound refers to a natural or synthetic compound that is capable of inducing an immune response when contacted with an immune cell expressing a functional TLR3 polypeptide. Preferably the immune response is or involves activation of a TLR3 signal transduction pathway. Thus immunostimulatory compounds identified and characterized using the methods of the invention specifically include TLR3 ligands, i.e., compounds which selectively bind to TLR3 and induce a TLR3 signal transduction pathway. Immunostimulatory compounds in general include but are not limited to nucleic acids, including oligonucleotides and polynucleotides; oligopeptides; polypeptides; lipids, including lipopolysaccharides; carbohydrates, including oligosaccharides and polysaccharides; and small molecules. Accordingly, a “test compound” refers to nucleic acids, including oligonucleotides and polynucleotides; oligopeptides; polypeptides; lipids, including lipopolysaccharides; carbohydrates, including oligosaccharides and polysaccharides; and small molecules. Test compounds include compounds with known biological activity as well as compounds without known biological activity.

A “reference immunostimulatory compound” refers to an immunostimulatory compound that characteristically induces an immune response when contacted with an immune cell expressing a functional TLR polypeptide. In the screening methods of the invention, the reference immunositmulatory compound is a natural or synthetic compound that that characteristically induces an immune response when contacted with an immune cell expressing a functional TLR3 polypeptide. Preferably the immune response is or involves activation of a TLR3 signal transduction pathway. Thus a reference immunostimulatory compound will characteristically induce a reference response mediated by a TLR3 signal transduction pathway when contacted with a functional TLR3 under suitable conditions. The reference response can be measured according to any of the methods described herein. Importantly, a reference immunostimulatory compound specifically includes a test compound identified as an immunostimulatory compound according to any one of the methods of the invention. Therefore a reference immunostimulatory compound can be a nucleic acid, including oligonucleotides and polynucleotides; an oligopeptide; a polypeptide; a lipid, including lipopolysaccharides; a carbohydrate, including oligosaccharides and polysaccharides; or a small molecule.

Small molecules include naturally occurring, synthetic, and semisynthetic organic and organometallic compounds with molecular weight less than about 1.5 kDa. Examples of small molecules include most drugs, subunits of polymeric materials, and analogs and derivatives thereof.

A “nucleic acid” as used herein with respect to test compounds and reference compounds used in the methods of the invention, shall refer to any polymer of two or more individual nucleoside or nucleotide units. Typically individual nucleoside or nucleotide units will include any one or combination of deoxyribonucleosides, ribonucleosides, deoxyribonucleotides, and ribonucleotides. The individual nucleotide or nucleoside units of the nucleic acid can be naturally occurring or not naturally occurring. For example, the individual nucleotide units can include deoxyadenosine, deoxycytidine, deoxyguanosine, thymidine, and uracil. In addition to naturally occurring 2′-deoxy and 2′-hydroxyl forms, individual nucleosides also include synthetic nucleosides having modified base moieties and/or modified sugar moieties, e.g., as described in Uhlmann E et al. (1990) Chem Rev 90:543-84. The linkages between individual nucleotide or nucleoside units can be naturally occurring or not naturally occurring. For example, the linkages can be phosphodiester, phosphorothioate, phosphorodithioate, phosphoramidate, as well as peptide linkages and other covalent linkages, known in the art, suitable for joining adjacent nucleoside or nucleotide units. The nucleic acid test compounds and nucleic acid reference compounds typically range in size from 3-4 units to a few tens of units, e.g., 18-40 units.

The substituted purines and pyrimidines of the ISNAs include standard purines and pyrimidines such as cytosine as well as base analogs such as C-5 propyne substituted bases. Wagner R W et al. (1996) Nat Biotechnol 14:840-4. Purines and pyrimidines include but are not limited to adenine, cytosine, guanine, thymine, 5-methylcytosine, 2-aminopurine, 2-amino-6-chloropurine, 2,6-diaminopurine, hypoxanthine, and other naturally and non-naturally occurring nucleobases, substituted and unsubstituted aromatic moieties.

Libraries of compounds that can be used as test compounds are available from various commercial suppliers, and they can be made to order using techniques well known in the art, including combinatorial chemistry techniques. Especially in combination with high throughput screening methods, such methods including in particular automated multichannel methods of screening, large libraries of test compounds can be screened according to the methods of the invention. Large libraries can include hundreds, thousands, tens of thousands, hundreds of thousands, and even millions of compounds.

Thus in preferred embodiments, the methods for screening test compounds can be performed on a large scale and with high throughput by incorporating, e.g., an array-based assay system and at least one automated or semi-automated step. For example, the assays can be set up using multiple-well plates in which cells are dispensed in individual wells and reagents are added in a systematic manner using a multiwell delivery device suited to the geometry of the multiwell plate. Manual and robotic multiwell delivery devices suitable for use in a high throughput screening assay are well known by those skilled in the art. Each well or array element can be mapped in a one-to-one manner to a particular test condition, such as the test compound. Readouts can also be performed in this multiwell array, preferably using a multiwell plate reader device or the like. Examples of such devices are well known in the art and are available through commercial sources. Sample and reagent handling can be automated to further enhance the throughput capacity of the screening assay, such that dozens, hundreds, thousands, or even millions of parallel assays can be performed in a day or in a week. Fully robotic systems are known in the art for applications such as generation and analysis of combinatorial libraries of synthetic compounds. See, for example, U.S. Pat. Nos. 5,443,791 and 5,708,158.

A “CpG nucleic acid” or a “CpG immunostimulatory nucleic acid” as used herein is a nucleic acid containing at least one unmethylated CpG dinucleotide (cytosine-guanine dinucleotide sequence, i.e. “CpG DNA” or DNA containing a 5′ cytosine followed by 3′ guanine and linked by a phosphate bond) and activates a component of the immune system. The entire CpG nucleic acid can be unmethylated or portions may be unmethylated but at least the C of the 5′ CG 3′ must be unmethylated.

In one embodiment a CpG nucleic acid is represented by at least the formula:

5′-N₁X₁CGX₂N₂-3′

wherein X₁ and X₂ are nucleotides, N is any nucleotide, and N₁ and N₂ are nucleic acid sequences composed of from about 0-25 N's each. In some embodiments X₁ is adenine, guanine, or thymine and/or X₂ is cytosine, adenine, or thymine. In other embodiments X₁ is cytosine and/or X₂ is guanine.

Examples of CpG nucleic acids according to the invention include but are not limited to those listed in Table 1.

TABLE 1

Exemplary CpG Nucleic Acids

| | |
|---|---|
| AACGTTCT | |
| AAGCGAAAATGAAATTGACT | SEQ ID NO:39 |
| ACCATGGACGAACTGTTTCCCCTC | SEQ ID NO:40 |
| ACCATGGACGACCTGTTTCCCCTC | SEQ lD NO:41 |
| ACCATGGACGAGCTGTTTCCCCTC | SEQ ID NO:42 |
| ACCATGGACGATCTGTTTCCCCTC | SEQ ID NO:43 |
| ACCATGGACGGTCTGTTTCCCCTC | SEQ ID NO:44 |
| ACCATGGACGTACTGTTTCCCCTC | SEQ ID NO:45 |
| ACCATGGACGTTCTGTTTCCCCTC | SEQ ID NO:46 |
| AGCGGGGGCGAGCGGGGGCG | SEQ lD NO:47 |
| AGCTATGACGTTCCAAGG | SEQ ID NO:48 |
| ATCGACTCTCGAGCGTTCTC | SEQ ID NO:49 |
| ATGACGTTCCTGACGTT | SEQ ID NO:50 |
| ATGGAAGGTCCAACGTTCTC | SEQ ID NO:51 |
| ATGGAAGGTCCAGCGTTCTC | SEQ ID NO:52 |
| ATGGACTCTCCAGCGTTCTC | SEQ ID NO:53 |
| ATGGAGGCTCCATCGTTCTC | SEQ ID NO:54 |
| CAACGTT | |
| CACGTTGAGGGGCAT | SEQ ID NO:55 |
| CAGGCATAACGGTTCCGTAG | SEQ ID NO:56 |
| CCAACGTT | |
| CTGATTTCCCCGAAATGATG | SEQ ID NO:57 |
| GAGAACGATGGACCTTCCAT | SEQ ID NO:58 |
| GAGAACGCTCCAGCACTGAT | SEQ ID NO:59 |
| GAGAACGCTCGACCTTCCAT | SEQ ID NO:60 |
| GAGAACGCTCGACCTTCGAT | SEQ ID NO:61 |
| GAGAACGCTGGACCTTCCAT | SEQ ID NO:62 |
| GATTGCCTGACGTCAGAGAG | SEQ ID NO:63 |
| GCATGACGTTGAGCT | SEQ ID NO:64 |
| GCGGCGGGCGGCGCGCGCCC | SEQ ID NO:65 |
| GCGTGCGTTGTCGTTGTCGTT | SEQ ID NO:66 |
| GCTAGACGTTAGCGT | SEQ ID NO:67 |
| GCTAGACGTTAGTGT | SEQ ID NO:68 |
| GCTAGATGTTAGCGT | SEQ ID NO:69 |
| GCTTGATGACTCAGCCGGAA | SEQ ID NO:70 |
| GGAATGACGTTCCCTGTG | SEQ ID NO:71 |
| GGGGTCAACGTTGACGGGG | SEQ ID NO:72 |
| GGGGTCAGTCTTGACGGGG | SEQ ID NO:73 |
| GTCCATTTCCCGTAAATCTT | SEQ ID NO:74 |
| GTCGCT | |
| GTCGTT | |
| TACCGCGTGCGACCCTCT | SEQ ID NO:75 |
| TCAACGTC | |
| TCAACGTT | |
| TCAGCGCT | |
| TCAGCGTGCGCC | SEQ ID NO:76 |
| TCATCGAT | |
| TCCACGACGTTTTCGACGTT | SEQ ID NO:77 |
| TCCATAACGTTCCTGATGCT | SEQ ID NO:78 |
| TCCATAGCGTTCCTAGCGTT | SEQ ID NO:79 |
| TCCATCACGTGCCTGATGCT | SEQ ID NO:80 |
| TCCATGACGGTCCTGATGCT | SEQ ID NO:81 |
| TCCATGACGTCCCTGATGCT | SEQ ID NO: 82 |
| TCCATGACGTGCCTGATGCT | SEQ ID NO:83 |
| TCCATGACGTTCCTGACGTT | SEQ ID NO:84 |
| TCCATGACGTTCCTGATGCT | SEQ ID NO:18 |
| TCCATGCCGGTCCTGATGCT | SEQ ID NO:85 |
| TCCATGCGTGCGTGCGTTTT | SEQ ID NO:86 |
| TCCATGCGTTGCGTTGCGTT | SEQ ID NO:87 |
| TCCATGGCGGTCCTGATGCT | SEQ ID NO:88 |
| TCCATGTCGATCCTGATGCT | SEQ ID NO:89 |
| TCCATGTCGCTCCTGATGCT | SEQ ID NO:90 |
| TCCATGTCGGTCCTGATGCT | SEQ ID NO:91 |
| TCCATGTCGGTCCTGCTGAT | SEQ ID NO:92 |
| TCCATGTCGTCCCTGATGCT | SEQ ID NO:93 |
| TCCATGTCGTTCCTGATGCT | SEQ ID NO:94 |
| TCCATGTCGTTCCTGTCGTT | SEQ ID NO:95 |
| TCCATGTCGTTTTTGTCGTT | SEQ ID NO:96 |
| TCCTGACGTTCCTGACGTT | SEQ ID NO:97 |
| TCCTGTCGTTCCTGTCGTT | SEQ ID NO:98 |
| TCCTGTCGTTCCTTGTCGTT | SEQ ID NO:99 |
| TCCTGTCGTTTTTTGTCGTT | SEQ ID NO:100 |
| TCCTTGTCGTTCCTGTCGTT | SEQ ID NO:101 |
| TCGATCGGGGCGGGGCGAGC | SEQ ID NO:102 |
| TCGTCGCTGTCTCCGCTTCTT | SEQ ID NO:103 |
| TCGTCGCTGTCTCCGCTTCTTCTTGCC | SEQ ID NO:104 |
| TCGTCGCTGTCTGCCCTTCTT | SEQ ID NO:105 |
| TCGTCGCTGTTGTCGTTTCTT | SEQ ID NO:106 |
| TCGTCGTCGTCGTT | SEQ ID NO:107 |
| TCGTCGTTGTCGTTGTCGTT | SEQ ID NO:108 |
| TCGTCGTTGTCGTTTTGTCGTT | SEQ ID NO:109 |
| TCGTCGTTTTGTCGTTTTGTCGTT | SEQ ID NO:15 |
| TCTCCCAGCGCGCGCCAT | SEQ ID NO:110 |
| TCTCCCAGCGGGCGCAT | SEQ ID NO:111 |
| TCTCCCAGCGTGCGCCAT | SEQ ID NO:112 |
| TCTTCGAA | |
| TGCAGATTGCGCAATCTGCA | SEQ ID NO:113 |
| TGTCGCT | |
| TGTCGTT | |
| TGTCGTTGTCGTT | SEQ ID NO:114 |
| TGTCGTTGTCGTTGTCGTT | SEQ ID NO: 115 |
| TGTCGTTGTCGTTGTCGTTGTCGTT | SEQ ID NO:116 |
| TGTCGTTTGTCGTTTGTCGTT | SEQ ID NO:117 |

As used herein the term “response mediated by a TLR signal transduction pathway” refers to a response which is characteristic of an interaction between a TLR and an immunostimulatory compound that induces signaling events through the TLR. Such responses typically involve usual elements of Toll/IL-1R signaling, e.g., MyD88, TRAF, and IRAK molecules, although in the case of TLR3 the role of MyD88 is less clear than for other TLR family members. As demonstrated herein such responses include the induction of a gene under control of a specific promoter such as a NF-κB promoter, increases in particular cytokine levels, increases in particular chemokine levels etc. The gene under the control of the NF-κB promoter may be a gene which naturally includes an NF-κB promoter or it may be a gene in a construct in which an NF-κB promoter has been inserted. Genes which naturally include the NF-κB promoter include but are not limited to IL-8, IL-12 p40, NF-κB-luc, IL-12 p40-luc, and TNF-luc. Increases in cytokine levels may result from increased production or increased stability or increased secretion of the cytokines in response to the TLR-immunostimulatory compound interaction. Th1 cytokines include but are not limited to IL-2, IFN-γ, and IL-12. It has unexpectedly been discovered, according to the instant invention, that the promoter response element ISRE is directly activated as a result of signaling through the TLR3 signal transduction pathway, i.e., independent of IFN-γ production. Th2 cytokines include but are not limited to IL-4, IL-5, and IL-10. Chemokines of particular significance in the invention include but are not limited to CCL5 (RANTES), CXCL9 (Mig), CXCL10 (IP-10), and CXCL11 (I-TAC).

In another aspect the invention provides a screening method for identifying a compound that modulates TLR3 signaling activity. The method according to this aspect of the invention involves the steps of (a) contacting a functional TLR3 with a test compound and a reference immunostimulatory compound under conditions which, in presence of the reference immunostimulatory compound alone, permit a reference response mediated by a TLR3 signal transduction pathway; (b) detecting a test-reference response mediated by the TLR3 signal transduction pathway; (c) determining the test compound is an agonist of TLR3 signaling activity when the test-reference response exceeds the reference response; and (d) determining the test compound is an antagonist of TLR3 signaling activity when the reference response exceeds the test-reference response. A test-reference response refers to a type of test response as determined when a test compound and a reference immunostimulatory compound are simultaneously contacted with the TLR3. When a test compound is neither an agonist nor an antagonist of TLR3 signaling activity, the test-reference response and the reference response are indistinguishable.

An agonist as used herein is a compound which causes an enhanced response of a TLR to a reference stimulus. The enhanced response can be additive or synergistic with respect to the response to the reference stimulus by itself. Furthermore, an agonist can work directly or indirectly to cause the enhanced response. Thus an agonist of TLR3 signaling activity as used herein is a compound which causes an enhanced response of a TLR to a reference stimulus.

An antagonist as used herein is a compound which causes a diminished response of a TLR to a reference stimulus. Furthermore, an antagonist can work directly or indirectly to cause the diminished response. Thus an antagonist of TLR3 signaling activity as used herein is a compound which causes a diminished response of a TLR to a reference stimulus.

In addition to identification and characterization of immunostimulatory compounds, agonists of TLR3 signaling, and antagonists of TLR3 signaling, the methods of the invention also permit optimization of lead compounds. Optimization of a lead compound involves an iterative application of a screening method of the invention, further including the steps of selecting the best candidate at any given stage or round in the screening and then substituting it as a benchmark or reference in a subsequent round of screening. This latter process can further include selection of parameters to modify in choosing and generating candidate test compounds to screen. For example, a lead compound from a particular round of screening can be used as a basis to develop a focused library of new test compounds for use in a subsequent round of screening.

In another aspect the invention provides a screening method for identifying species specificity of an immunostimulatory compound. The method according to this aspect of the invention involves the steps of (a) measuring a first species-specific response mediated by a TLR3 signal transduction pathway when a functional TLR3 of a first species is contacted with a test compound; (b) measuring a second species-specific response mediated by the TLR3 signal transduction pathway when a functional TLR3 of a second species is contacted with the test compound; and (c) comparing the first species-specific response with the second species-specific response.

A species-specific TLR, including TLR3, is not limited to a human TLR, but rather can include a TLR derived from human or non-human sources. Examples of non-human sources include, but are not limited to, murine, rat, bovine, canine, feline, ovine, porcine, and equine. Other species include chicken and fish, e.g., aquaculture species.

The species-specific TLR, including TLR3, also is not limited to native TLR polypeptides. In certain embodiments the TLR can be, e.g., a chimeric TLR in which the extracellular domain and the cytoplasmic domain are derived from TLR polypeptides from different species. Such chimeric TLR polypeptides, as described above, can include, for example, a human TLR extracellular domain and a murine TLR cytoplasmic domain, each domain derived from the corresponding TLR of each species. In alternative embodiments, such chimeric TLR polypeptides can include chimeras created with different TLR splice variants or allotypes. Other chimeric TLR polypeptides useful for the screening methods of the invention include chimeric polypeptides created with a TLR of a first type, e.g., TLR3, and another TLR, e.g., TLR7, TLR8, or TLR9, of the same or another species as the TLR of the first type. Also contemplated are chimeric polypeptides which incorporate sequences derived from more than two polypeptides, e.g., an extracellular domain, a transmembrane domain, and a cytoplasmic domain all derived from different polypeptide sources, provided at least one such domain derives from a TLR3 polypeptide. As a further example, also contemplated are constructs such as include an extracellular domain of one TLR3, an intracellular domain of another TLR3, and a non-TLR reporter such as luciferase, GFP, etc. Those of skill in the art will recognize how to design and generate DNA sequences coding for such chimeric TLR polypeptides.

It has also been discovered, according to the instant invention, that TLR-based screening assays, including but not limited to the TLR3-based assays described herein, are sensitive to parameters such as concentration of test compound, stability of test compound, kinetics of detection, and selection of reporter. These parameters can be optimized in order to derive the most information from a given screening assay. Importantly, the kinetics of detection appear to afford separation of types of information such as affinity of interaction and stability or duration of interaction. For example, measurements taken at earlier timepoints, e.g., after 6-8 hours of contact between TLR and test and/or reference compound, appear to reflect more information about affinity of interaction than do measurements obtained at later timepoints, e.g., after 16-24 or more hours of contact. In addition, while NF-κB-driven reporters are generally useful in TLR-based screening assays like those of the instant invention, in some instances a reporter other than an NF-κB-driven reporter will afford greater sensitivity. For example, the IL-8-luc reporter is significantly more sensitive to TLR7 and TLR8 than NF-κB-luc. Selection of reporter thus appears to be TLR-dependent, while parameters relating to kinetics and concentration appear to be more compound-dependent. Thus in performing the screening methods of the instant invention, it is expected that the methods will be enhance by inclusion of measurements obtained using at least two concentrations and two time points for each test compound. Typically at least three concentrations will be employed, spanning a two to three log-fold range of concentrations. Finer ranges of concentration can of course be employed under suitable circumstances, for instance based on results of an earlier screening performed using a wider initial range of concentrations.

The invention will be more fully understood by reference to the following examples. These examples, however, are merely intended to illustrate certain embodiments of the invention and are not to be construed to limit the scope of the invention.

### EXAMPLES

### Example 1

### Expression Vectors for Human TLR3 (hTLR3) and Murine TLR3 (mTLR3)

To create an expression vector for human TLR3, human TLR3 cDNA was amplified by the polymerase chain method (PCR) from a cDNA made from human 293 cells using the primers 5′-GAAACTCGAGCCACCATGAGACAGACTTTGCCTTGTATCTAC-3′ (sense, SEQ ID NO:9) and 5′-GAAAGAATTCTTAATGTACAGAGTTTTTGGATCCAAG-3′ (antisense, SEQ ID NO:10). The primers introduce Xho I and EcoRI restriction endonuclease sites at their 5′ ends for use in subsequent cloning into the expression vector. The resulting amplication product fragment was cloned into pGEM-T Easy vector (Promega), isolated, cut with Xho I and EcoRI restriction endonucleases, ligated into an Xho I/EcoRI-digested pcDNA3.1 expression vector (Invitrogen). The insert was fully sequenced and translated into protein. The cDNA sequence corresponds to the published cDNA sequence for hTLR3, available as GenBank accession no. NM_{—}003265 (SEQ ID NO:1). The open reading frame codes for a protein 904 amino acids long, having the sequence corresponding to GenBank accession no. NP_{—}003256 (SEQ ID NO:2).

TABLE 2

cDNA Sequence for Human TLR3

(GenBank Accession No. NM 003265: SEQ ID NO:1)

| | |
|---|---|
| gcggccgcgt cgacgaaatg tctggatttg gactaaagaa aaaaggaaag gctagcagtc | 60 |
| atccaacaga atcatgagac agactttgcc ttgtatctac ttttgggggg gccttttgcc | 120 |
| ctttgggatg ctgtgtgcat cctccaccac caagtgcact gttagccatg aagttgctga | 180 |
| ctgcagccac ctgaagttga ctcaggtacc cgatgatcta cccacaaaca taacagtgtt | 240 |
| gaaccttacc cataatcaac tcagaagatt accagccgcc aacttcacaa ggtatagcca | 300 |
| gctaactagc ttggatgtag gatttaacac catctcaaaa ctggagccag aattgtgcca | 360 |
| gaaacttccc atgttaaaag ttttgaacct ccagcacaat gagctatctc aactttctga | 420 |
| taaaaccttt gccttctgca cgaatttgac tgaactccat ctcatgtcca actcaatcca | 480 |
| gaaaattaaa aataatccct ttgtcaagca gaagaattta atcacattag atctgtctca | 540 |
| taatggcttg tcatctacaa aattaggaac tcaggttcag ctggaaaatc tccaagagct | 600 |
| tctattatca aacaataaaa ttcaagcgct aaaaagtgaa gaactggata tctttgccaa | 660 |
| ttcatcttta aaaaaattag agttgtcatc gaatcaaatt aaagagtttt ctccagggtg | 720 |
| ttttcacgca attggaagat tatttggcct ctttctgaac aatgtccagc tgggtcccag | 780 |
| ccttacagag aagctatgtt tggaattagc aaacacaagc attcggaatc tgtctctgag | 840 |
| taacagccag ctgtccacca ccagcaatac aactttcttg ggactaaagt ggacaaatct | 900 |
| cactatgctc gatctttcct acaacaactt aaatgtggtt ggtaacgatt cctttgcttg | 960 |
| gcttccacaa ctagaatatt tcttcctaga gtataataat atacagcatt tgttttctca | 1020 |
| ctctttgcac gggcttttca atgtgaggta cctgaatttg aaacggtctt ttactaaaca | 1080 |
| aagtatttcc cttgcctcac tccccaagat tgatgatttt tcttttcagt ggctaaaatg | 1140 |
| tttggagcac cttaacatgg aagataatga tattccaggc ataaaaagca atatgttcac | 1200 |
| aggattgata aacctgaaat acttaagtct atccaactcc tttacaagtt tgcgaacttt | 1260 |
| gacaaatgaa acatttgtat cacttgctca ttctccctta cacatactca acctaaccaa | 1320 |
| gaataaaatc tcaaaaatag agagtgatgc tttctcttgg ttgggccacc tagaagtact | 1380 |
| tgacctgggc cttaatgaaa ttgggcaaga actcacaggc caggaatgga gaggtctaga | 1440 |
| aaatattttc gaaatctatc tttcctacaa caagtacctg cagctgacta ggaactcctt | 1500 |
| tgccttggtc ccaagccttc aacgactgat gctccgaagg gtggccctta aaaatgtgga | 1560 |
| tagctctcct tcaccattcc agcctcttcg taacttgacc attctggatc taagcaacaa | 1620 |
| caacatagcc aacataaatg atgacatgtt ggagggtctt gagaaactag aaattctcga | 1680 |
| tttgcagcat aacaacttag cacggctctg gaaacacgca aaccctggtg gtcccattta | 1740 |
| tttcctaaag ggtctgtctc acctccacat ccttaacttg gagtccaacg gctttgacga | 1800 |
| gatcccagtt gaggtcttca aggatttatt tgaactaaag atcatcgatt taggattgaa | 1860 |
| taatttaaac acacttccag catctgtctt taataatcag gtgtctctaa agtcattgaa | 1920 |
| ccttcagaag aatctcataa catccgttga gaagaaggtt ttcgggccag ctttcaggaa | 1980 |
| cctgactgag ttagatatgc gctttaatcc ctttgattgc acgtgtgaaa gtattgcctg | 2040 |
| gtttgttaat tggattaacg agacccatac caacatccct gagctgtcaa gccactacct | 2100 |
| ttgcaacact ccacctcact atcatgggtt cccagtgaga ctttttgata catcatcttg | 2160 |
| caaagacagt gccccctttg aactcttttt catgatcaat accagtatcc tgttgatttt | 2220 |
| tatctttatt gtacttctca tccactttga gggctggagg atatcttttt attggaatgt | 2280 |
| ttcagtacat cgagttcttg gtttcaaaga aatagacaga cagacagaac agtttgaata | 2340 |
| tgcagcatat ataattcatg cctataaaga taaggattgg gtctgggaac atttctcttc | 2400 |
| aatggaaaag gaagaccaat ctctcaaatt ttgtctggaa gaaagggact ttgaggcggg | 2460 |
| tgtttttgaa ctagaagcaa ttgttaacag catcaaaaga agcagaaaaa ttatttttgt | 2520 |
| tataacacac catctattaa aagacccatt atgcaaaaga ttcaaggtac atcatgcagt | 2580 |
| tcaacaagct attgaacaaa atctggattc cattatattg gttttccttg aggagattcc | 2640 |
| agattataaa ctgaaccatg cactctgttt gcgaagagga atgtttaaat ctcactgcat | 2700 |
| cttgaactgg ccagttcaga aagaacggat aggtgccttt cgtcataaat tgcaagtagc | 2760 |
| acttggatcc aaaaactctg tacattaaat ttatttaaat attcaattag caaaggagaa | 2820 |
| actttctcaa tttaaaaagt tctatggcaa atttaagttt tccataaagg tgttataatt | 2880 |
| tgtttattca tatttgtaaa tgattatatt ctatcacaat tacatctctt ctaggaaaat | 2940 |
| gtgtctcctt atttcaggcc tatttttgac aattgactta attttaccca aaataaaaca | 3000 |
| tataagcacg caaaaaaaaa aaaaaaaaa 3029 | |

TABLE 3

Amino Acid Sequence for Human TLR3

(GenBank Accession No. NP 003256; SEQ ID NO:2)

| | |
|---|---|
| MRQTLPCIYF WGGLLPFGML CASSTTKCTV SHEVADCSHL KLTQVPDDLP TNITVLNLTH | 60 |
| NQLRRLPAAN FTRYSQLTSL DVGFNTISKL EPELCQKLPM LKVLNLQHNE LSQLSDKTFA | 120 |
| FCTNLTELHL MSNSIQKIKN NPFVKQKNLI TLDLSHNGLS STKLGTQVQL ENLQELLLSN | 180 |
| NKIQALKSEE LDIFANSSLK KLELSSNQIK EFSPGCFHAI GRLFGLFLNN VQLGPSLTEK | 240 |
| LCLELANTSI RNLSLSNSQL STTSNTTFLG LKWTNLTMLD LSYNNLNVVG NDSFAWLPQL | 300 |
| EYFFLEYNNI QHLFSHSLHG LFNVRYLNLK RSFTKQSISL ASLPKIDDFS FQWLKCLEHL | 360 |
| NMEDNDIPGI KSNMFTGLIN LKYLSLSNSF TSLRTLTNET FVSLAHSPLH ILNLTKNKIS | 420 |
| KIESDAFSWL GHLEVLDLGL NEIGQELTGQ EWRGLENIFE IYLSYNKYLQ LTRNSFALVP | 480 |
| SLQRLMLRRV ALKNVDSSPS PFQPLRNLTI LDLSNNNIAN INDDMLEGLE KLEILDLQHN | 540 |
| NLARLWKHAN PGGPIYFLKG LSHLHILNLE SNGFDEIPVE VFKDLFELKI IDLGLNNLNT | 600 |
| LPASVFNNQV SLKSLNLQKN LITSVEKKVF GPAFRNLTEL DMRFNPFDCT CESIAWFVNW | 660 |
| INETHTNIPE LSSHYLCNTP PHYHGFPVRL FDTSSCKDSA PFLEFFMINT SILLIFIFIV | 720 |
| LLIHFEGWRI SFYWNVSVHR VLGFKEIDRQ TEQFEYAAYI IHAYKDKDWV WEHFSSMEKE | 780 |
| DQSLKFCLEE RDFEAGVFEL EAIVNSIKRS RKIIFVITHH LLKDPLCKRF KVHHAVQQAI | 840 |
| EQNLDSIILV FLEEIPDYKL NHALCLRRGM FKSHCILNWP VQKERIGAFR HKLQVALGSK | 900 |
| NSVH 904 | |

Corresponding nucleotide and amino acid sequences for murine TLR3 (mTLR3) are known. The nucleotide sequence of mTLR3 cDNA has been reported as GenBank accession no. AF355152, and the amino acid sequence of mTLR3 has been reported as GenBank accession no. AAK26117.

TABLE 4
cDNA Sequence for Murine TLR3

(GenBank Accession No. AF355152; SEQ ID NO:3)

| | |
|---|---|
| tagaatatga tacagggatt gcacccataa tctgggctga atcatgaaag ggtgttcctc | 60 |
| ttatctaatg tactcctttg ggggactttt gtccctatgg attcttctgg tgtcttccac | 120 |
| aaaccaatgc actgtgagat acaacgtagc tgactgcagc catttgaagc taacacacat | 180 |
| acctgatgat cttccctcta acataacagt gttgaatctt actcacaacc aactcagaag | 240 |
| attaccacct accaacttta caagatacag ccaacttgct atcttggatg caggatttaa | 300 |
| ctccatttca aaactggagc cagaactgtg ccaaatactc cctttgttga aagtattgaa | 360 |
| cctgcaacat aatgagctct ctcagatttc tgatcaaacc tttgtcttct gcacgaacct | 420 |
| gacagaactc gatctaatgt ctaactcaat acacaaaatt aaaagcaacc ctttcaaaaa | 480 |
| ccagaagaat ctaatcaaat tagatttgtc tcataatggt ttatcatcta caaagttggg | 540 |
| aacgggggtc caactggaga acctccaaga actgctctta gcaaaaaata aaatccttgc | 600 |
| gttgcgaagt gaagaacttg agtttcttgg caattcttct ttacgaaagt tggacttgtc | 660 |
| atcaaatcca cttaaagagt tctccccggg gtgtttccag acaattggca agttattcgc | 720 |
| cctcctcttg aacaacgccc aactgaaccc ccacctcaca gagaagcttt gctgggaact | 780 |
| ttcaaacaca agcatccaga atctctctct ggctaacaac cagctgctgg ccaccagcga | 840 |
| gagcactttc tctgggctga agtggacaaa tctcacccag ctcgatcttt cctacaacaa | 900 |
| cctccatgat gtcggcaacg gttccttctc ctatctccca agcctgaggt atctgtctct | 960 |
| ggagtacaac aatatacagc gtctgtcccc tcgctctttt tatggactct ccaacctgag | 1020 |
| gtacctgagt ttgaagcgag catttactaa gcaaagtgtt tcacttgctt cacatcccaa | 1080 |
| cattgacgat ttttcctttc aatggttaaa atatttggaa tatctcaaca tggatgacaa | 1140 |
| taatattcca agtaccaaaa gcaatacctt cacgggattg gtgagtctga agtacctaag | 1200 |
| tctttccaaa actttcacaa gtttgcaaac tttaacaaat gaaacatttg tgtcacttgc | 1260 |
| tcattctccc ttgctcactc tcaacttaac gaaaaatcac atctcaaaaa tagcaaatgg | 1320 |
| tactttctct tggttaggcc aactcaggat acttgatctc ggccttaatg aaattgaaca | 1380 |
| aaaactcagc ggccaggaat ggagaggtct gagaaatata tttgagatct acctatccta | 1440 |
| taacaaatac ctccaactgt ctaccagttc ctttgcattg gtccccagcc ttcaaagact | 1500 |
| gatgctcagg agggtggccc ttaaaaatgt ggatatctcc ccttcacctt tccgccctct | 1560 |
| tcgtaacttg accattctgg acttaagcaa caacaacata gccaacataa atgaggactt | 1620 |
| gctggagggt cttgagaatc tagaaatcct ggattttcag cacaataact tagccaggct | 1680 |
| ctggaaacgc gcaaaccccg gtggtcccgt taatttcctg aaggggctgt ctcacctcca | 1740 |
| catcttgaat ttagagtcca acggcttaga tgaaatccca gtcggggttt tcaagaactt | 1800 |
| attcgaacta aagagcatca atctaggact gaataactta aacaaacttg aaccattcat | 1860 |
| ttttgatgac cagacatctc taaggtcact gaacctccag aagaacctca taacatctgt | 1920 |
| tgagaaggat gttttcgggc cgccttttca aaacctgaac agtttagata tgcgcttcaa | 1980 |
| tccgttcgac tgcacgtgtg aaagtatttc ctggtttgtt aactggatca accagaccca | 2040 |
| cactaatatc tttgagctgt ccactcacta cctctgtaac actccacatc attattatgg | 2100 |
| cttccccctg aagcttttcg atacatcatc ctgtaaagac agcgccccct ttgaactcct | 2160 |
| cttcataatc agcaccagta tgctcctggt ttttatactt gtggtactgc tcattcacat | 2220 |
| cgagggctgg aggatctctt tttactggaa tgtttcagtg catcggattc ttggtttcaa | 2280 |
| ggaaatagac acacaggctg agcagtttga atatacagcc tacataattc atgcccataa | 2340 |
| agacagagac tgggtctggg aacatttctc cccaatggaa gaacaagacc attctctcaa | 2400 |
| attttgccta gaagaaaggg actttgaagc aggcgtcctt ggacttgaag caattgttaa | 2460 |
| tagcatcaaa agaagccgaa aaatcatttt cgttatcaca caccatttat taaaagaccc | 2520 |
| tctgtgcaga agattcaagg tacatcacgc agttcagcaa gctattgagc aaaatctgga | 2580 |
| ttcaattata ctgatttttc tccagaatat tccagattat aaactaaacc atgcactctg | 2640 |
| tttgcgaaga ggaatgttta aatctcattg catcttgaac tggccagttc agaaagaacg | 2700 |
| gataaatgcc tttcatcata aattgcaagt agcacttgga tctcggaatt cagcacatta | 2760 |
| aactcatttg aagatttgga gtcggtaaag ggatagatcc aatttataaa ggtccatcat | 2820 |
| gaatctaagt tttacttgaa agttttgtat atttatttat atgtatagat gatgatatta | 2880 |
| catcacaatc caatctcagt tttgaaatat ttcggcttat ttcattgaca tctggtttat | 2940 |
| tcactccaaa taaacacatg ggcagttaaa aacatcctct attaatagat tacccattaa | 3000 |
| ttcttgaggt gtatcacagc tttaaagggt tttaaatatt tttatataaa taagactgag | 3060 |
| agttttataa atgtaatttt ttaaaactcg agtcttactg tgtagctcag aaaggcctgg | 3120 |
| aaattaatat attagagagt catgtcttga acttatttat ctctgcctcc ctctgtctcc | 3180 |
| agagtgttgc ttttaagggc atgtagcacc acacccagct atgtacgtgt gggattttat | 3240 |
| aatgctcatt tttgagacgt ttatagaata aaagataatt gcttttatgg tataaggcta | 3300 |
| cttgaggtaa 3310 | |

TABLE 5

Amino Acid Sequence for Murine TLR3

(GenBank Accession No. AAK26117; SEQ ID NO:4)

| | |
|---|---|
| MKGCSSYLMY SFGGLLSLWI LLVSSTNQCT VRYNVADCSH LKLTHIPDDL PSNITVLNLT | 60 |
| HNQLRRLPPT NFTRYSQLAI LDAGFNSISK LEPELCQILP LLKVLNLQHN ELSQISDQTF | 120 |
| VFCTNLTELD LMSNSIHKIK SNPFKNQKNL IKLDLSHNGL SSTKLGTGVO LENLQELLLA | 180 |
| KNKILALRSE ELEFLGNSSL RKLDLSSNPL KEFSPGCFQT IGKLFALLLN NAQLNPHLTE | 240 |
| KLCWELSNTS IQNLSLANNQ LLATSESTFS GLKQTNLTQL DLSYNNLHDV GNGSFSYLPS | 300 |
| LRYLSLEYNN IQRLSPRSFY GLSNLRYLSL KRAFTKQSVS LASHPNIDDF SFQWLKYLEY | 360 |
| LNMDDNNIPS TKSNTFTGLV SLKYLSLSKT FTSLQTLTNE TFVSLAHSPL LTLNLTKNHI | 420 |
| SKISNGTFSW LGQLRILDLG LNEIEQKLSG QEWRGLRNIF EIYLSYNKYL QLSTSSFALV | 480 |
| PSLQRLMLRR VALKNVDISP SPFRPLRNLT ILDLSNNNIA NINEDLLEGL ENLEILDFQH | 540 |
| NNLARLWKRA NPGGPVNFLK GLSHLHILNL ESNGLDEIPV GVFKNLFELF SINLGLNNLN | 600 |
| KLEPFIFDDQ TSLRSLNLQK NLITSVEKDV FGPPFQNLNS LDMRFNPFDC TCESISWFVN | 660 |
| WINQTHTNIF ELSTHYLCNT PHHYYGFPLK LFDTSSCKDS APFELLFIIS TSMLLVFILV | 720 |
| VLLIHIEGWR ISFYWNVSVH RILGFKEIDT QARQFEYTAY IIHAHKDRDW VWEHFSPMEE | 780 |
| QDQSLKFCLE ERDFEAGVLG LEAIVNSIKR SRKIIFVITH HLLKDPLCRR FKVHHAVQQA | 840 |
| IEQNLDSIIL IFLQNIPDYK LNHALCLRRG MFKSHCILNW PVQKERINAF HHKLQVALGS | 900 |
| RNSAH 905 | |

### Example 2

### Method of Making IFN-α4 Reporter Vector

A number of reporter vectors may be used in the practice of the invention. Some of the reporter vectors are commercially available, e.g., the luciferase reporter vectors pNF-κB-Luc (Stratagene) and pAP1-Luc (Stratagene). These two reporter vectors place the luciferase gene under control of an upstream (5′) promoter region derived from genomic DNA for NF-κB or AP1, respectively. Other reporter vectors can be constructed following standard methods using the desired promoter and a vector containing a suitable reporter, such as luciferase, β-galactosidase (β-gal), chloramphenicol acetyltransferase (CAT), and other reporters known by those skilled in the art. Following are some examples of reporter vectors constructed for use in the present invention.

IFN-α4 is an immediate-early type 1 IFN. Sequence-specific PCR products for the −620 to +50 promoter region of IFN-α4 were derived from genomic DNA of human 293 cells and cloned into SmaI site of the pGL3-Basic Vector (Promega). The resulting expression vector includes a luciferase gene under control of an upstream (5′) −620 to +50 promoter region of IFN-α4. The sequence of the −620 to +50 promoter region of IFN-α4 is provided as SEQ ID NO:11 in Table 6.

TABLE 6

Nucleotide Sequence of the −620 to +50 Promoter Region of
Human IFN-α4

(SEQ ID NO:11)

| | |
|---|---|
| agaaaaattt taaaaaatta ttcattcata tttttaggag ttttgaatga ttggatatgt | 60 |
| aattatattc atattattaa tgtgtatcta tatagatttt tattttgcat atgtactttg | 120 |
| atacaaaatt tacatgaaca aattacacta aaagttattc cacaaatata cttatcaaat | 180 |
| taagttaaat gtcaatagct tttaaactta aattttagtt taacttttct gtcattcttt | 240 |
| actttgaata aaaagagcaa actttgtagt ttttatctgt gaagtagagg tatacgtaat | 300 |
| atacataaat agatatgcca aatctgtgtt attaaaattt catgaagatt tcaattagaa | 360 |
| aaaaatacca taaaaggctt tgagtgcagg tgaaaaatag gcaatgatga aaaaaaatga | 420 |
| aaaacttttt aaacacatgt agagagtgcg taaagaaagc aaaaacagag atagaaagta | 480 |
| caactaggga atttagaaaa tggaaattag tatgttcact atttaagacc tatgcacaga | 540 |
| gcaaagtctt cagaaaacct agaggccgaa gttcaaggtt atccatctca agtagcctag | 600 |
| caatatttgc aacatcccaa tggccctgtc cttttcttta ctgatggccg tgctggtgct | 660 |
| cagctacaaa 670 | |

### Example 3

### Method of Making IFN-α1 Reporter Vector

IFN-α1 is a late type 1 IFN. Sequence-specific PCR products for the −140 to +9 promoter region of IFN-α1 were derived from genomic DNA of human 293 cells and cloned into SmaI site of the pGL3-Basic Vector (Promega). The resulting expression vector includes a luciferase gene under control of an upstream (5′) −140 to +9 promoter region of IFN-α1.

### Example 4

### Method of Making IFN-β Reporter Vector

IFN-β is an immediate-early type 1 IFN. The −280 to +20 promoter region of IFN-β was derived from the pUCβ26 vector (Algarté M et al. (1999) J Virol 73(4):2694-702) by restriction at EcoRI and TaqI sites. The 300 bp restriction fragment was filled in by Klenow enzyme and cloned into NheI-digested and filled in pGL3-Basic Vector (Promega). The resulting expression vector includes a luciferase gene under control of an upstream (5′) −280 to +20 promoter region of IFN-β. The sequence of the −280 to +20 promoter region of IFN-β is provided as SEQ ID NO:12 in Table 7.

TABLE 7

Nucleotide Sequence of the −280 to +20 Promoter Region of
Human IFn-β

(SEQ ID NO:12)

| | |
|---|---|
| ttctcaggtc gtttgctttc ctttgctttc tcccaagtct tgttttacaa tttgctttag | 60 |
| tcattcactg aaactttaaa aaacattaga aaacctcaca gtttgtaaat ctttttccct | 120 |
| attatatata tcataagata ggagcttaaa taaagagttt tagaaactac taaaatgtaa | 180 |
| atgacatagg aaaactgaaa gggagaagtg aaagtgggaa attcctctga atagagagag | 240 |
| gaccatctca tataaatagg ccatacccac ggagaaagga cattctaact gcaacctttc | 300 |

### Example 5

### Method of Making RANTES Reporter Vector

Transcription of the chemokine RANTES is believed to be regulated at least in part by IRF3 and by NF-κB. Lin R et al. (1999) J Mol Cell Biol 19(2):959-66; Genin P et al. (2000) J Immunol 164:5352-61. A 483 bp sequence-specific PCR product including the −397 to +5 promoter region of RANTES was derived from genomic DNA of human 293 cells, restricted with PstI and cloned into pCAT-Basic Vector (Promega) using HindIII (filled in with Klenow) and PstI sites (filled in). The −397 to +5 promoter region of RANTES was then isolated from the resulting RANTES/chloramphenicol acetyltransferase (CAT) reporter plasmid by restriction with BglII and SalI, filled in with Klenow enzyme, and cloned into the NheI site (filled in with Klenow) of the pGL3-Basic Vector (Promega). The resulting expression vector includes a luciferase gene under control of an upstream (5′) −397 to +5 promoter region of RANTES. Comparison of the insert sequence −397 to +5 of Genin P et al. (2000) J Immunol 164:5352-61 and GenBank accession no. AB023652 (SEQ ID NO:13) revealed two point deletions (at positions 105 and 273 of SEQ ID NO:13) which do not create new restriction sites. The sequence of the −397 to +5 promoter region of RANTES is provided as SEQ ID NO:14 in Table 8.

TABLE 8

Nucleotide Sequence of the −397 to +5 Promoter Region of
Human RANTES

SEQ ID NO:14)

| | |
|---|---|
| gatctgtaat gaataagcag gaactttgaa gactcagtga ctcagtgagt aataaagact | 60 |
| cagtgacttc tgatcctgtc ctaactgcca ctccttgttg tcccaagaaa gcggcttcct | 120 |
| gctctctgag gaggacccct tccctggaag gtaaaactaa ggatgtcagc agagaaattt | 180 |
| ttccaccatt ggtgcttggt caaagaggaa actgatgagc tcactctaga tgagagagca | 240 |
| gtgagggaga gacagagact cgaatttccg gagctatttc agttttcttt tccgttttgt | 300 |
| gcaatttcac ttatgatacc ggccaatgct tggttgctat tttggaaact ccccttaggg | 360 |
| gatgcccctc aactggccct ataaagggcc agcctgagct g 401 | |

TABLE 9
Nucleotide Sequence of GenBank Accession No. AB023652

(SEQ ID NO:13)

| | |
|---|---|
| agaaggcctt acagtgagat gggatcccag tatttattga gtttcctcat tcataaaatg | 60 |
| gggataataa tagtaaatga gttgacacgc gctaagacag tggaatagtg gctggcacag | 120 |
| ataagccctc ggtaaatggt agccaataat gatagagtat gctgtaagat atctttctct | 180 |
| ccctctgctt ctcaacaagt ctctaatcaa ttattccact ttataaacaa ggaaatagaa | 240 |
| ctcaaagaca ttaagcactt ttcccaaagg tcgcttagca agtaaatggg agagacccta | 300 |
| tgaccaggat gaaagcaaga aattcccaca agaggactca ttccaactca tatcttgtga | 360 |
| aaaggttccc aatgcccagc tcagatcaac tgcctcaatt tacagtgtga gtgtgctcac | 420 |
| ctcctttggg gactgtatat ccagaggacc ctcctcaata aaacacttta taaataacat | 480 |
| ccttccatgg atgagggaaa ggaggtaaga tctgtaatga ataagcagga actttgaaga | 540 |
| ctcagtgact cagtgagtaa taaagactca gtgacttctg atcctgtcct aactgccact | 600 |
| ccttgttgtc cccaagaaag cggcttcctg ctctctgagg aggacccctt ccctggaagg | 660 |
| taaaactaag gatgtcagca gagaaatttt tccaccattg gtgcttggtc aaagaggaaa | 720 |
| ctgatgagct cactctagat gagagagcag tgagggagag acagagactc gaatttccgg | 780 |
| aggctatttc agttttcttt tccgttttgt gcaatttcac ttatgatacc ggccaatgct | 840 |
| tggttgctat tttggaaact ccccttaggg gatgcccctc aactggccct ataaagggcc | 900 |
| agcctgagct gcagaggatt cctgcagagg atcaagacag cacgtggacc tcgcacagcc | 960 |
| tctcccacag gtaccatgaa ggtctccgcg gcagccctcg ctgtcatcct cattgctact | 1020 |
| gccctctgcg c 1031 | |

### Example 6

### Method of Making Human IL-12 p40 Reporter Vectors

Reporter constructs have been made using truncated (−250 to +30) and full length (−860 to +30) promoter regions derived from human IL-12 p40 genomic DNA. In one reporter construct the truncated IL-12 p40 promoter was cloned as a KpnI-XhoI insert into pβgal-Basic (Promega). The resulting expression vector includes a β gal gene under control of an upstream (5′) −250 to +30 promoter region of human IL-12 p40. In a second reporter construct the full length IL-12 p40 promoter was cloned as a KpnI-XhoI insert into pβgal-Basic (Promega). The resulting expression vector includes a β gal gene under control of an upstream (5′) −860 to +30 promoter region of human IL-12 p40. In a third reporter construct the truncated −250 to +30 promoter region of human IL-12 p40 was cloned into the pGL3-Basic Vector (Promega). The resulting expression vector includes a luciferase gene under control of an upstream (5′) −250 to +30 promoter region of human IL-12 p40. In a fourth reporter construct the full length IL-12 p40 promoter of human IL-12 p40 was cloned into the pGL3-Basic Vector (Promega). The resulting expression vector includes a luciferase gene under control of an upstream (5′) −860 to +30 promoter region of human IL-12 p40.

### Example 7

### Method of Making Human IL-6 Reporter Vectors

Reporter constructs are made using the −235 to +7 promoter region derived from human IL-6 genomic DNA. In one reporter construct the IL-6 promoter region is cloned as a KpnI-XhoI insert into pGL3-Basic Vector (Promega). The resulting expression vector includes a luciferase gene under control of an upstream (5′) −235 to +7 promoter region derived from human IL-6 genomic DNA.

### Example 8

### Method of Making Human IL-8 Reporter Vectors

Reporter constructs have been made using a −546 to +44 and a truncated −133 to +44 promoter region derived from human IL-8 genomic DNA. Mukaida N et al. (1989) J Immunol 143:1366-71. In each reporter construct the IL-8 promoter region was cloned as a KpnI-XhoI insert into pGL3-Basic Vector (Promega). One of the resulting expression vectors includes a luciferase gene under control of an upstream (5′) −546 to +44 promoter region derived from human IL-8 genomic DNA. Another of the resulting expression vectors includes a luciferase gene under control of an upstream (5′) −133 to +44 promoter region derived from human IL-8 genomic DNA.

### Example 9

### Sequence Comparison of Human TLR3 and Human TLR9

Human TLR3 and TLR9 are homologous proteins with several structural commonalities. Both appear to be transmembrane proteins with an extracellular domain and an intracellular domain. Common characteristics include a signal sequence and transmembranal domain. Similarities common to most TLRs include a cysteine rich domain and a TIR domain. Most TLRs have leucine rich repeats (LRR) in their extracellular domain. TLR3, TLR7, TLR8, and TLR9 appear to have similar structures. The regularity of the leucine repeats are shown below for TLR3 and TLR9. These four TLRs can be broken into two extracellular subdomains, domain 1 and 2, by virtue of a separation by an unstructured hinge region. TLR7, TLR8, and TLR9 have 14 LRR in domain 1 and 12 LRR in domain 2. TLR9 is a known nucleic acid binder, interacting with CpG-DNA. It has been suspected that TLR7 and TLR8 most likely also interact with nucleic acids. TLR3 has a similar 11 LRR in domain 1 and has 12 LRR in domain 2, lacking the initial 3 repeats common to TLR7, TLR8, and TLR9. Based on structural consideration it is hypothesized that TLR3 interacts with nucleic acids or similar structures.

The structure of TLR3 differs from TLR7, TLR8, and TLR9 in an interesting character. Referring to Table 13, within the TIR domain it has been shown that a proline (shown in bold) is required for MyD88 interaction. MyD88 is required for TLR9 to transduce signal for the activation of NF-κB. Both TLR7 and TLR8 also have this proline. TLR3 however has an alanine at this position (also shown in bold). It is believed by the applicant that this difference may disallow MyD88 interaction with TLR3 and thus result in an altered signal transduction pattern compared to, e.g., TLR9.

TABLE 10
Sequence Alignment of hTLR9 (SEQ ID NO:6)
and hTLR3 (SEQ ID NO:2)
SIGNAL SEQUENCE

| | | |
|---|---|---|
| hTLR9 | MGFCRSALHPLSLLVQAIMLAMTLALGTLPAFLPCELQPHGLVNCNW | 47 |
| hTLR3 | MRQTLPCIYFWGGLLPFGMLCASSTTKCTVSHEVADC | 37 |

DOMAIN 1 LEUCINE RICH REPEATS

| | | |
|---|---|---|
| hTLR9 | LFLKSVPHFSMAAPRGNVTSLSLSSN | 73 |
| hTLR9 | RIHHLHDSDFAHLPSLRHLNLKWN | 97 |
| hTLR9 | CPPVGLSPMHFPCHMTIEPSTFLAVPTLEELNLSYN | 133 |
| hTLR9 | NIMTVPALPKSLISLSLSHT | 153 |
| hTLR3 | SHLKLTQVPDDLPTNITVLNLTHN | 61 |
| hTLR9 | NILMLDSASLAGLHALRFLFMDGN | 177 |
| hTLR3 | QLRRLPAANFTRYSQLTSLDVGFN | 85 |
| hTLR9 | CYYKNPCRQALEVAPGALLGLGNLTHLSLKYN | 209 |
| hTLR3 | TISKLEPELCQKLPMLKVLNLQHN | 109 |
| hTLR9 | NLTVVPRNLPSSLEYLLLSYN | 230 |
| hTLR3 | ELSQLSDKTFAFCTNLTELHLMSN | 133 |
| hTLR9 | RIVKLAPEDLANLTALRVLDVGGN | 254 |
| hTLR3 | SIQKIKNNPFVKQKNLITLDLSHN | 157 |
| hTLR9 | CRRCDHAPNPCMECPRHFPQLHPDTFSHLSRLEGLVLKDS | 294 |
| hTLR3 | GLSSTKLGTQVQLENLQELLLSNN | 181 |
| hTLR9 | SLSWLNASWFRGLGNLRVLDLSEN | 318 |
| hTLR3 | KIQALKSEELDIFANSSLKKLELSSN | 207 |
| hTLR9 | FLYKCITKTKAFQGLTQLRKLNLSFN | 344 |
| hTLR3 | QIKEFSPGCFHAIGRLFGLFLNNV | 231 |
| hTLR9 | YQKRVSFAHLSLAPSFGSLVALKELDMHGI | 374 |
| hTLR3 | QLGPSLTEKLCLELANTSIRNLSLSNS | 258 |
| hTLR9 | FFRSLDETTLRPLARLPMLQTLRLQMN | 401 |
| hTLR3 | QLSTTSNTTFLGLKWTNLTMLDLSYN | 284 |
| hTLR9 | FINQAQLGIFRAFPGLRYVDLSDN | 425 |
| hTLR3 | NLNVVGNDSFAWLPQLEYFFLEYN | 308 |

HINGE REGION

| | | |
|---|---|---|
| hTLR9 | RISGASELTATMGEADGGEKVWLQPGDLAPAPV | 458 |
| hTLR3 | NIQHLFSHSLHGLFNVRYLNLKRSFTKQSISLA | 341 |

DOMAIN 2 LEUCINE RICH REPEATS

| | | |
|---|---|---|
| hTLR9 | DTPSSEDFRPNCSTLNFTLDLSRN | 482 |
| hTLR3 | SLPKIDDFSFQWLKCLEHLNMEDN | 365 |
| hTLR9 | NLVTVQPEMFAQLSHLQCLRLSHN | 506 |
| hTLR3 | DIPGIKSNMFTGLINLKYLSLSNS | 389 |
| hTLR9 | CISQAVNGSQFLPLTGLQVLDLSHN | 531 |
| hTLR3 | FTSLRTLTNETFVSLAHSPLHILNLTKN | 417 |
| hTLR9 | KLDLYHEHSFTELPRLEALDLSYN | 555 |
| hTLR3 | KISKIESDAFSWLGHLEVLDLGLN | 441 |
| hTLR9 | SQPFGMQGVGHNFSFVAHLRTLRHLSLAHN | 585 |
| hTLR3 | EIGQELTGQEWRGLENIFEIYLSYN | 466 |
| hTLR9 | NIHSQVSQQLCSTSLRALDFSGN | 608 |
| hTLR3 | KYLQLTRNSFALVPSLQRLMLRRV | 490 |
| hTLR9 | ALGHMWAEGDLYLHFFQGLSGLIWLDLSQN | 638 |
| hTLR3 | ALKNVDSSPSPFQPLRNLTILDLSNN | 516 |
| hTLR9 | RLHTLLPQTLRNLPKSLQVLRLRDN | 663 |
| hTLR3 | NIANINDDMLEGLEKLEILDLQHN | 540 |
| hTLR9 | YLAFFKWWSLHFLPKLEVLDLAGN | 687 |
| hTLR3 | NLARLWKHANPGGPIYFLKGLSHLHILNLESN | 572 |
| hTLR9 | QLKALTNGSLPAGTRLRRLDVSCN | 711 |
| hTLR3 | GFDEIPVEVFKDLFELKIIDLGLN | 596 |
| hTLR9 | SISFVAPGFFSKAKELRELNLSAN | 735 |
| hTLR3 | NLNTLPASVFNNQVSLKSLNLQKN | 620 |
| hTLR9 | ALKTVDHSWFGPLASALQILDVSAN | 760 |
| hTLR3 | LITSVEKKVFGPAFRNLTELDMRFN | 645 |

CYSTEINE RICH DOMAIN

| | | |
|---|---|---|
| hTLR9 | PLHCACG**AAFMDFLLEVQAAVPGLPSRVKCGSPGQLQGLSIFAQD | 805 |
| hTLR3 | PFDCTCESIAWFVNWINETHTNIPELSSHYLCNTPPHYHGFPVRLFD | 692 |
| hTLR9 | LRLCLDEALSWDCFA | 820 |
| hTLR3 | TSSCKDSAPFELFFM | 707 |

TRANSMEMBRANAL DOMAIN

| | | |
|---|---|---|
| hTLR9 | LSLLAVALGLGVPMLHHL | 838 |
| hTLR3 | INTSILLIFIFIVLLIHF | 725 |

TIR DOMAIN

| | | |
|---|---|---|
| hTLR9 | CGWDLWYCFHLCLAWLPWRGRQSGRDEDALPYDAFVVFDKTQSAVAD | 885 |
| hTLR3 | EGWRISFYWNVSVHRVLGFKEIDRQTEQFE*YAAYIIHAYK***DKD | 768 |
| hTLR9 | WVYNELRGQLEECRGRWALRLCLEERDWLPGKTLFENLWASVYGSRK | 932 |
| hTLR3 | WVW***EHFSSMEKEDQSLKFCLEERDFEAGVFELEAIVNSIKRSRK | 812 |
| hTLR9 | TLFVLAHTD*RVSGLLRASFLLAQQRLLEDRKDVVVLVILSPDGRRS | 978 |
| hTLR3 | IIFVITHHLLKDPLCKRFKVHHAVQQAIEQNLDSIILVFLEEIPDYK | 859 |
| hTLR9 | ***RYVRLRQRLCRQSVLLWPHQPSGQRSFWAQLGMALTRDNHHFYN | 1022 |
| hTLR3 | LNHALCLRRGMFKSHCILNWPVQKERIGAFRHKLQVALGSKNSVH | 904 |
| hTLR9 | RNFCQGPTAE | 1032 |

### Example 10

### Reconstitution of TLR9 Signaling in 293 Fibroblasts

Methods for cloning murine and human TLR9 have been described in pending U.S. patent application Ser. No. 09/954,987 and corresponding published PCT application PCT/US01/29229, both filed Sep. 17, 2001, the contents of which are incorporated by reference. Human TLR9 cDNA and murine TLR9 cDNA in pT-Adv vector (from Clonetech) were individually cloned into the expression vector pcDNA3.1(−) from Invitrogen using the EcoRI site. Utilizing a “gain of function” assay it was possible to reconstitute human TLR9 (hTLR9) and murine TLR9 (mTLR9) signaling in CpG-DNA non-responsive human 293 fibroblasts (ATCC, CRL-1573). The expression vectors mentioned above were transfected into 293 fibroblast cells using the calcium phosphate method.

TABLE 11

cDNA Sequence for Human TLR9

(GenBank Accession No. AF245704; SEQ ID NO:5)

| | |
|---|---|
| aggctggtat aaaaatctta cttcctctat tctctgagcc gctgctgccc ctgtgggaag | 60 |
| ggacctcgag tgtgaagcat ccttccctgt agctgctgtc cagtctgccc gccagaccct | 120 |
| ctggagaagc ccctgccccc cagcatgggt ttctgccgca gcgccctgca cccgctgtct | 180 |
| ctcctggtgc aggccatcat gctggccatg accctggccc tgggtacctt gcctgccttc | 240 |
| ctaccctgtg agctccagcc ccacggcctg gtgaactgca actggctgtt cctgaagtct | 300 |
| gtgccccact tctccatggc agcaccccgt ggcaatgtca ccagcctttc cttgtcctcc | 360 |
| aaccgcatcc accacctcca tgattctgac tttgcccacc tgcccagcct gcggcatctc | 420 |
| aacctcaagt ggaactgccc gccggttggc ctcagcccca tgcacttccc ctgccacatg | 480 |
| accatcgagc ccagcacctt cttggctgtg cccaccctgg aagagctaaa cctgagctac | 540 |
| aacaacatca tgactgtgcc tgcgctgccc aaatccctca tatccctgtc cctcagccat | 600 |
| accaacatcc tgatgctaga ctctgccagc ctcgccggcc tgcatgccct gcgcttccta | 660 |
| ttcatggacg gcaactgtta ttacaagaac ccctgcaggc aggcactgga ggtggccccg | 720 |
| ggtgccctcc ttggcctggg caacctcacc cacctgtcac tcaagtacaa caacctcact | 780 |
| gtggtgcccc gcaacctgcc ttccagcctg gagtatctgc tgttgtccta caaccgcatc | 840 |
| gtcaaactgg cgcctgagga cctggccaat ctgaccgccc tgcgtgtgct cgatgtgggc | 900 |
| ggaaattgcc gccgctgcga ccacgctccc aacccctgca tggagtgccc tcgtcacttc | 960 |
| ccccagctac atcccgatac cttcagccac ctgagccgtc ttgaaggcct ggtgttgaag | 1020 |
| gacagttctc tctcctggct gaatgccagt tggttccgtg ggctgggaaa cctccgagtg | 1080 |
| ctggacctga gtgagaactt cctctacaaa tgcatcacta aaaccaaggc cttccagggc | 1140 |
| ctaacacagc tgcgcaagct taacctgtcc ttcaattacc aaaagagggt gtcctttgcc | 1200 |
| cacctgtctc tggccccttc cttcgggagc ctggtcgccc tgaaggagct ggacatgcac | 1260 |
| ggcatcttct tccgctcact cgatgagacc acgctccggc cactggcccg cctgcccatg | 1320 |
| ctccagactc tgcgtctgca gatgaacttc atcaaccagg cccagctcgg catcttcagg | 1380 |
| gccttccctg gcctgcgcta cgtggacctg tcggacaacc gcatcagcgg agcttcggag | 1440 |
| ctgacagcca ccatggggga ggcagatgga ggggagaagg tctggctgca gcctggggac | 1500 |
| cttgctccgg ccccagtgga cactcccagc tctgaagact tcaggcccaa ctgcagcacc | 1560 |
| ctcaacttca ccttggatct gtcacggaac aacctggtga ccgtgcagcc ggagatgttt | 1620 |
| gcccagctct cgcacctgca gtgcctgcgc ctgagccaca actgcatctc gcaggcagtc | 1680 |
| aatggctccc agttcctgcc gctgaccggt ctgcaggtgc tagacctgtc ccgcaataag | 1740 |
| ctggacctct accacgagca ctcattcacg gagctaccgc gactggaggc cctggacctc | 1800 |
| agctacaaca gccagccctt tggcatgcag ggcgtgggcc acaacttcag cttcgtggct | 1860 |
| cacctgcgca ccctgcgcca cctcagcctg gcccacaaca acatccacag ccaagtgtcc | 1920 |
| cagcagctct gcagtacgtc gctgcgggcc ctggacttca gcggcaatgc actgggccat | 1980 |
| atgtgggccg agggagacct ctatctgcac ttcttccaag gcctgagcgg tttgatctgg | 2040 |
| ctggacttgt cccagaaccg cctgcacacc ctcctgcccc aaaccctgcg caacctcccc | 2100 |
| aagagcctac aggtgctgcg tctccgtgac aattacctgg ccttctttaa gtggtggagc | 2160 |
| ctccacttcc tgcccaaact ggaagtcctc gacctggcag gaaaccggct gaaggccctg | 2220 |
| accaatggca gcctgcctgc tggcacccgg ctccggaggc tggatgtcag ctgcaacagc | 2280 |
| atcagcttcg tggcccccgg cttcttttcc aaggccaagg agctgcgaga gctcaacctt | 2340 |
| agcgccaacg ccctcaagac agtggaccac tcctggtttg ggcccctggc gagtgccctg | 2400 |
| caaatactag atgtaagcgc caaccctctg cactgcgcct gtggggcggc ctttatggac | 2460 |
| ttcctgctgg aggtgcaggc tgccgtgccc ggtctgccca gccgggtgaa gtgtggcagt | 2520 |
| ccgggccagc tccagggcct cagcatcttt gcacaggacc tgcgcctctg cctggatgag | 2580 |
| gccctctcct gggactgttt cgccctctcg ctgctggctg tggctctggg cctgggtgtg | 2640 |
| cccatgctgc atcacctctg tggctgggac ctctggtact gcttccacct gtgcctggcc | 2700 |
| tggcttccct ggcgggggcg gcaaagtggg cgagatgagg atgccctgcc ctacgatgcc | 2760 |
| ttcgtggtct tcgacaaaac gcagagcgca gtggcagact gggtgtacaa cgagcttcgg | 2820 |
| gggcagctgg aggagtgccg tgggcgctgg gcactccgcc tgtgcctgga ggaacgcgac | 2880 |
| tggctgcctg gcaaaaccct ctttgagaac ctgtgggcct cggtctatgg cagccgcaag | 2940 |
| acgctgtttg tgctggccca cacggaccgg gtcagtggtc tcttgcgcgc cagcttcctg | 3000 |
| ctggcccagc aqcgcctgct ggaggaccgc aaggacgtcg tggtgctggt gatcctgagc | 3060 |
| cctgacggcc gccgctcccg ctacgtgcgg ctgcgccagc gcctctgccg ccagagtgtc | 3120 |
| ctcctctggc cccaccagcc cagtggtcag cgcagcttct gggcccagct gggcatggcc | 3180 |
| ctgaccaggg acaaccacca cttctataac cggaacttct gccagggacc cacggccgaa | 3240 |
| tagccgtgag ccggaatcct gcacggtgcc acctccacac tcacctcacc tctgcctgcc | 3300 |
| tggtctgacc ctcccctgct cgcctccctc accccacacc tgacacagag ca 3352 | |

TABLE 12

Amino Acid Sequence for Human TLR9

(GenBank Accession No. AAF78037, SEQ ID NO:6)+HZ,1/44

| | |
|---|---|
| MGFCRSALHP LSLLVQAIML AMTLALGTLP AFLPCELQPH GLVNCNWLFL KSVPHFSMAA | 60 |
| PRGNVTSLSL SSNRIHHLHD SDFAHLPSLR HLNLKWNCPP VGLSPMHFPC HMTIEPSTFL | 120 |
| AVPTLEELNL SYNNIMTVPA LPKSLISLSL SHTNILMLDS ASLAGLHALR FLFMDGNCYY | 180 |
| KNPCRQALEV APGALLGLGN LTHLSLKYNN LTVVPRNLPS SLEYLLLSYN RIVKLAPEDL | 240 |
| ANLTALRVLD VGGNCRRCDH APNPCMECPR HFPQLHPDTF SHLSRLEGLV LKDSSLSWLN | 300 |
| ASWFRGLGNL RVLDLSENFL YKCITKTKAF QGLTQLRKLM LSFNYQKRVS FAHLSLAPSF | 360 |
| GSLVALKELD MHGIFFRSLD ETTLRPLARL PMLQTLRLQM NFINQAQLGI FRAFPGLRYV | 420 |
| DLSDNRISGA SELTATMGEA DGGEKVWLQP GDLAPAPVDT PSSEDFRPNC STLNFTLDLS | 480 |
| RNNLVTVQPE MFAQLSHLQC LRLSHNCISQ AVNGSQFLPL TGLQVLDLSR NKLDLYHEHS | 540 |
| FTELPRLEAL DLSYNSQPFG MQGVGHNFSF VAHLRTLRHL SLAHNNTHSQ VSQQLCSTSL | 600 |
| RALDFSGNAL GHMWAEGDLY LHFFQGLSGL IWLDLSQNRL HTLLPQTLRN LPKSLQVLRL | 660 |
| RDNYLAFFKW WSLHFLPKLE VLDLAGNRLK ALTNGSLPAG TRLRRLDVSC NSISFVAPGF | 720 |
| FSKAKELREL NLSANALKTV DHSWFGPLAS ALQILDVSAN PLHCACGAAF MDFLLEVQAA | 780 |
| VPGLPSRVKC GSPGQLQGLS IFAQDLRLCL DEALSWDCFA LSLLAVALCL GVPMLHHLCG | 840 |
| WDLWYCFHLC LAWLPWRGRQ SGRDEDALPY DAFVVFDKTQ SAVADWVYNE LRGQLEECRG | 900 |
| RWALRLCLEE RDWLPGKTLF ENLWASVYGS RKTLFVLAHT DRVSGLLRAS FLLAQQRLLE | 960 |
| DRKDVVVLVI LSPDGRRSRY VRLRQRLCRQ SVLLWPHQPS GQRSFWAQLG MALTRDNHHF | 1020 |
| YNRNFCQGPT AE 1032 | |

TABLE 13

cDNA Sequence for Murine TLR9
(GenBank Accession No. AF348140; SEQ ID NO:7)

| | |
|---|---|
| tgtcagaggg agcctcggga gaatcctcca tctcccaaca tggttctccg tcgaaggact | 60 |
| ctgcacccct tgtccctcct ggtacaggct gcagtgctgg ctgagactct ggccctgggt | 120 |
| accctgcctg ccttcctacc ctgtgagctg aagcctcatg gcctggtgga ctgcaattgg | 180 |
| ctgttcctga agtctgtacc ccgtttctct gcggcagcat cctgctccaa catcacccgc | 240 |
| ctctccttga tctccaaccg tatccaccac ctgcacaact ccgacttcgt ccacctgtcc | 300 |
| aacctgcggc agctgaacct caagtggaac tgtccaccca ctggccttag ccccctgcac | 360 |
| ttctcttgcc acatgaccat tgagcccaga accttcctgg ctatgcgtac actggaggag | 420 |
| ctgaacctga gctataatgg tatcaccact gtgccccgac tgcccagctc cctggtgaat | 480 |
| ctgagcctga gccacaccaa catcctggtt ctagatgcta acagcctcgc cggcctatac | 540 |
| agcctgcgcg ttctcttcat ggacgggaac tgctactaca agaacccctg cacaggagcg | 600 |
| gtgaaggtga ccccaggcgc cctcctgggc ctgagcaatc tcacccatct gtctctgaag | 660 |
| tataacaacc tcacaaaggt gccccgccaa ctgcccccca gcctggagta cctcctggtg | 720 |
| tcctataacc tcattgtcaa gctggggcct gaagacctgg ccaatctgac ctcccttcga | 780 |
| gtacttgatg tgggtgggaa ttgccgtcgc tgcgaccatg cccccaatcc ctgtatagaa | 840 |
| tgtggccaaa agtccctcca cctgcaccct gagaccttcc atcacctgag ccatctggaa | 900 |
| ggcctggtgc tgaaggacag ctctctccat acactgaact cttcctggtt ccaaggtctg | 960 |
| gtcaacctct cggtgctgga cctaagcgag aactttctct atgaaagcat caaccacacc | 1020 |
| aatgcctttc agaacctaac ccgcctgcgc aagctcaacc tgtccttcaa ttaccgcaag | 1080 |
| aaggtatcct ttgcccgcct ccacctggca agttccttca agaacctggt gtcactgcag | 1140 |
| gagctgaaca tgaacggcat cttcttccgc tcgctcaaca agtacacgct cagatggctg | 1200 |
| gccgatctgc ccaaactcca cactctgcat cttcaaatga acttcatcaa ccaggcacag | 1260 |
| ctcagcatct ttggtacctt ccgagccctt cgctttgtgg acttgtcaga caatcgcatc | 1320 |
| agtgggcctt caacgctgtc agaagccacc cctgaagagg cagatgatgc agagcaggag | 1380 |
| gagctgttgt ctgcggatcc tcacccagct ccactgagca cccctgcttc taagaacttc | 1440 |
| atggacaggt gtaagaactt caagttcacc atggacctgt ctcggaacaa cctggtgact | 1500 |
| atcaagccag agatgtttgt caatctctca cgcctccagt gtcttagcct gagccacaac | 1560 |
| tccattgcac aggctgtcaa tggctctcag ttcctgccgc tgactaatct gcaggtgctg | 1620 |
| gacctgtccc ataacaaact ggacttgtac cactggaaat cgttcagtga gctaccacag | 1680 |
| ttgcaggccc tggacctgag ctacaacagc cagcccttta gcatgaaggg tataggccac | 1740 |
| aatttcagtt ttgtggccca tctgtccatg ctacacagcc ttagcctggc acacaatgac | 1800 |
| attcataccc gtgtgtcctc acatctcaac agcaactcag tgaggtttct tgacttcagc | 1860 |
| ggcaacggta tgggccgcat gtgggatgag gggggccttt atctccattt cttccaaggc | 1920 |
| ctgagtggcc tgctgaagct ggacctgtct caaaataacc tgcatatcct ccggccccag | 1980 |
| aaccttgaca acctccccaa gagcctgaag ctgctgagcc tccgagacaa ctacctatct | 2040 |
| ttctttaact ggaccagtct gtccttcctg cccaacctgg aagtcctaga cctggcaggc | 2100 |
| aaccagctaa aggccctgac caatggcacc ctgcctaatg gcaccctcct ccagaaactg | 2160 |
| gatgtcagca gcaacagtat cgtctctgtg gtcccagcct tcttcgctct ggcggtcgag | 2220 |
| ctgaaagagg tcaacctcag ccacaacatt ctcaagacgg tggatcgctc ctggtttggg | 2280 |
| cccattgtga tgaacctgac agttctagac gtgagaagca accctctgca ctgtgcctgt | 2340 |
| ggggcagcct tcgtagactt actgttggag gtgcagacca aggtgcctgg cctggctaat | 2400 |
| ggtgtgaagt gtggcagccc cggccagctg cagggccgta gcatcttcgc acaggacctg | 2460 |
| cggctgtgcc tggatgaggt cctctcttgg gactgctttg gcctttcact cttggctgtg | 2520 |
| gccgtgggca tggtggtgcc tatactgcac catctctgcg gctgggacgt ctggtactgt | 2580 |
| tttcatctgt gcctggcatg gctacctttg ctggcccgca gccgacgcag cgcccaagct | 2640 |
| ctcccctatg atgccttcgt ggtgttcgat aaggcacaga gcgcagttgc ggactgggtg | 2700 |
| tataacgagc tgcgggtgcg gctggaggag cggcgcggtc gccgagccct acgcttgtgt | 2760 |
| ctggaggacc gagattggct gcctggccag acgctcttcg agaacctctg ggcttccatc | 2820 |
| tatgggagcc gcaagactct atttgtgctg gcccacacgg accgcgtcag tggcctcctg | 2880 |
| cgcaccagct tcctgctggc tcagcagcgc ctgttggaag accgcaagga cgtggtggtg | 2940 |
| ttggtgatcc tgcgtccgga tgcccaccgc tcccgctatg tgcgactgcg ccagcgtctc | 3000 |
| tgccgccaga gtgtgctctt ctggccccag cagcccaacg ggcagggggg cttctgggcc | 3060 |
| cagctgagta cagccctgac tagggacaac cgccacttct ataaccagaa cttctgccgg | 3120 |
| ggacctacag cagaatagct cagagcaaca gctggaaaca gctgcatctt catgcctggt | 3180 |
| tcccgagttg ctctgcctgc 3200 | |

TABLE 14

Amino Acid Sequence for Murine TLR9

(GenBank Accession No. AAK29625; SEQ ID NO:8)

| | |
|---|---|
| MVLRRRTLHP LSLLVQAAVL AETLALGTLP AFLPCELKPH GLVDCNWLFL KSVPRFSAAA | 60 |
| SCSNITRLSL ISNRIHHLHN SDFVHLSNLR QLNLKWNCPP TGLSPLHFSC HMTIEPRTFL | 120 |
| AMRTLEELNL SYNGITTVPR LPSSLVNLSL SHTNILVLDA NSLAGLYSLR VLFMDGNCYY | 180 |
| KNPCTGAVKV TPGALLGLSN LTHLSLKYNN LTKVPRQLPP SLEYLLVSYN LIVKLGPEDL | 240 |
| ANLTSLRVLD VGGNCRRCDH APNPCIECGQ KSLHLHPETF HHLSHLEGLV LKDSSLHTLN | 300 |
| SSWFQGLVNL SVLDLSENFL YESTNBTNAF QNLTRLRKLN LSFNYRKKVS FARLHLASSF | 360 |
| KNLVSLQELN MNGIFFRSLN KYTLRWLADL PKLHTLHLQM NFINQAQLSI FGTFRALRFV | 420 |
| DLSDNRISGP STLSEATPEE ADDAEQEELL SADPHPAPLS TPASKNFMDR CKIFKFTMDL | 480 |
| SRNNLVTIKP EMFVNLSRLQ CLSLSHNSIA QAVNGSQFLP LTNLQVLDLS HNKLDLYHWK | 540 |
| SFSELPQLQA LDLSYNSQPF SMKGIGHNFS FVAHLSMLHS LSLAHNDIHT RVSSHLNSNS | 600 |
| VRFLDFSGNG MGRMWDEGGL YLHFFQGLSG LLKLDLSQNN LHILRPQNLD NLPKSLKLLS | 660 |
| LRDNYLSFFN WTSLSFLPNL EVLDLAGNQL KALTNGTLPN GTLLQKLDVS SNSIVSVVPA | 720 |
| FFALAVELKE VNLSHNTLKT VDRSWFGPTV MNLTVLDVRS NPLHCACGAA FVDLLLEVQT | 780 |
| KVPGLANGVK CGSPGQLQGR SIFAQDLRLC LDEVLSWDCF GLSLLAVAVG MVVPILHHLC | 840 |
| GWDVWYCFHL CLAWLPLLAR SRRSAQALPY DAFVVFDKAQ SAVADWVYNE LRVRLEERRG | 900 |
| RRALRLCLED RDWLPGQTLF ENLWASIYGS RKTLFVLAHT DRVSGLLRTS FLLAQQRLLE | 960 |
| DRKDVVVLVI LRPDAHRSRY VRLRQRLCRQ SVLFWPQQPN GQOGFWAQLS TALTRDNRHF | 1020 |
| YNQNFCRGPT AE 1032 | |

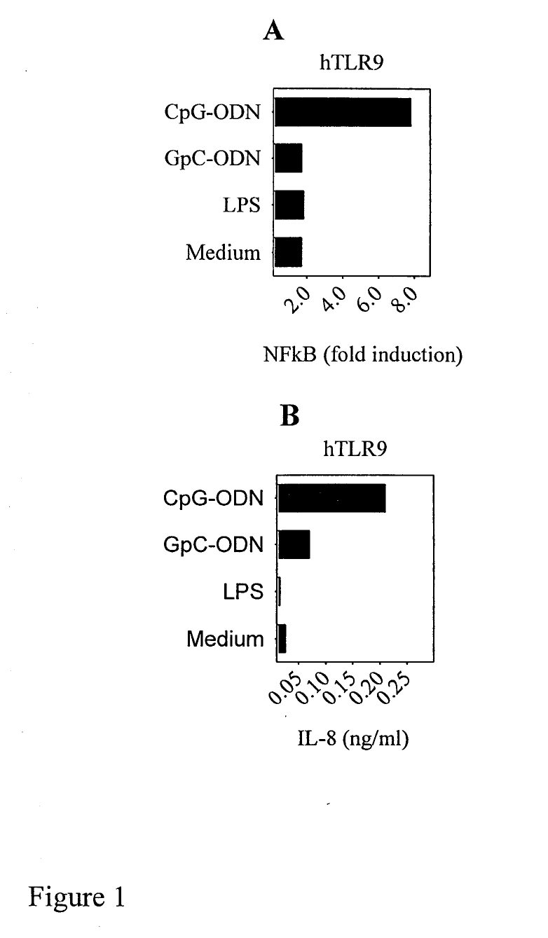

Since NF-κB activation is central to the IL-1/TLR signal transduction pathway (Medzhitov R et al. (1998) Mol Cell 2:253-258 (1998); Muzio M et al. (1998) J Exp Med 187:2097-101), cells were transfected with hTLR9 or co-transfected with hTLR9 and an NF-κB-driven luciferase reporter construct. Human 293 fibroblast cells were transiently transfected with (FIG. 1A) hTLR9 and a six-times NF-κB-luciferase reporter plasmid (NF-κB-luc, kindly provided by Patrick Baeuerle, Munich, Germany) or (FIG. 1B) with hTLR9 alone. After stimulus with CpG-ODN (2006, 2 μM, TCGTCGTTTTGTCGTTTTGTCGTT, SEQ ID NO:15), GpC-ODN (2006-GC, 2 μM, TGCTGCTTTTGTGCTTTTGTGCTT, SEQ ID NO:16), LPS (100 ng/ml) or media, NF-κB activation by luciferase readout (8 h, FIG. 1A) or IL-8 production by ELISA (48 h, FIG. 1B) were monitored. Results are representative of three independent experiments. FIG. 1 shows that cells expressing hTLR9 responded to CpG-DNA but not to LPS.

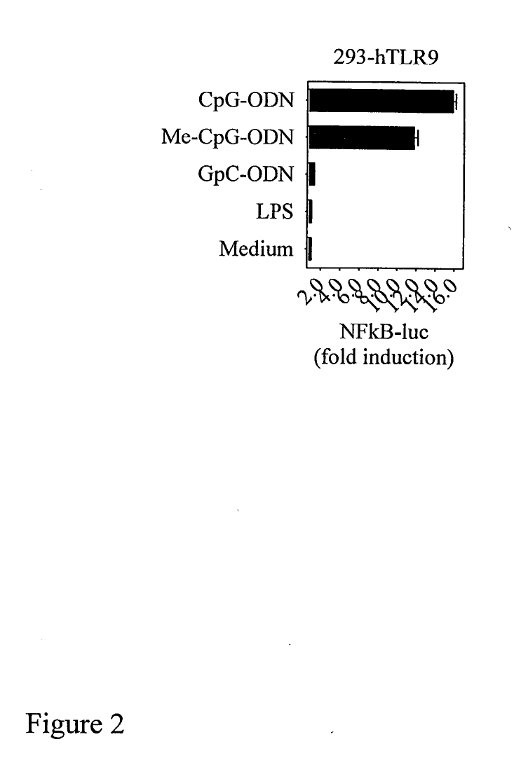

FIG. 2 demonstrates the same principle for the transfection of mTLR9. Human 293 fibroblast cells were transiently transfected with mTLR9 and the NF-κB-luc construct (FIG. 2). Similar data was obtained for IL-8 production (not shown). Thus expression of TLR9 (human or mouse) in 293 cells results in a gain of function for CpG-DNA stimulation similar to hTLR4 reconstitution of LPS responses.

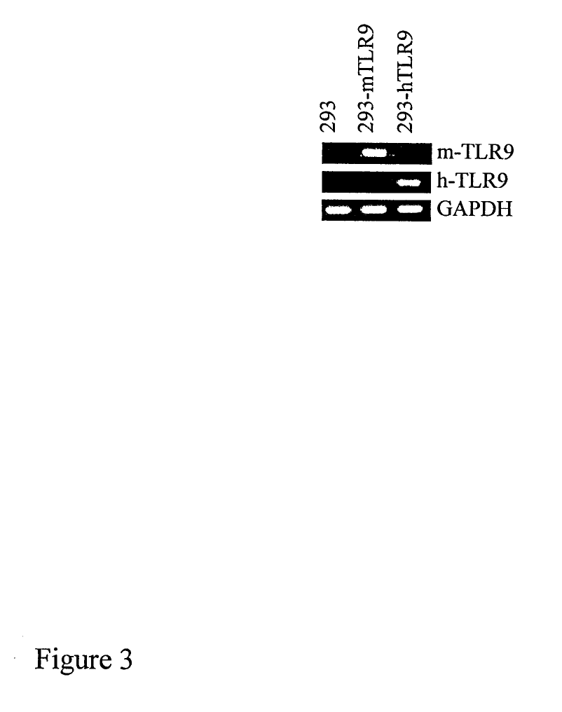

To generate stable clones expressing human TLR9, murine TLR9, or either TLR9 with the NF-κB-luc reporter plasmid, 293 cells were transfected in 10 cm plates (2×10⁶ cells/plate) with 16 μg of DNA and selected with 0.7 mg/ml G418 (PAA Laboratories GmbH, Cölbe, Germany). Clones were tested for TLR9 expression by RT-PCR, for example as shown in FIG. 3. The clones were also screened for IL-8 production or NF-κB-luciferase activity after stimulation with ODN. Four different types of clones were generated.

| | |
|---|---|
| 293-hTLR9-luc: | expressing human TLR9 and |
| | 6-fold NF-κB-luciferase reporter |
| 293-mTLR9-luc: | expressing murine TLR9 and |
| | 6-fold NF-κB-luciferase reporter |
| 293-hTLR9: | expressing human TLR9 |
| 293-mTLR9: | expressing murine TLR9 |

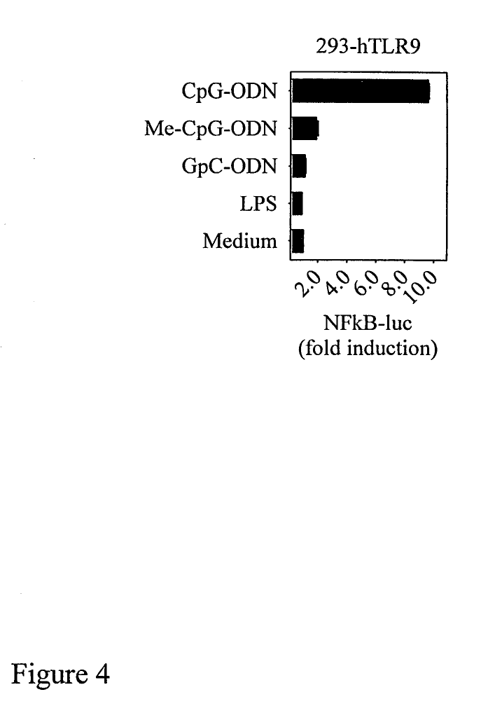

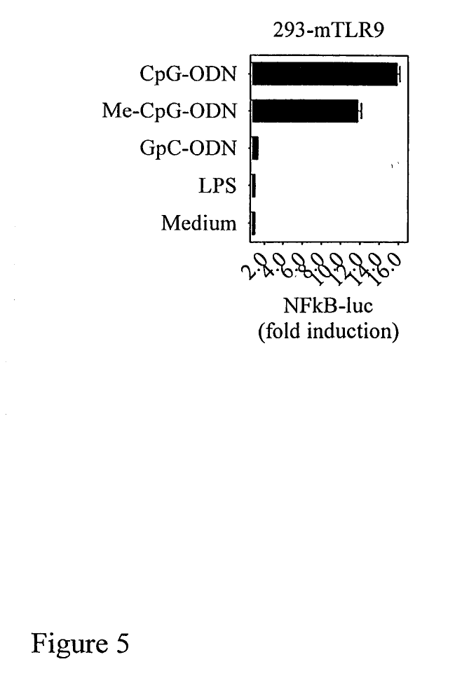

FIG. 4 demonstrates the responsiveness of a stable 293-hTLR9-luc clone after stimulation with CpG-ODN (2006, 2 μM), GpC-ODN (2006-GC, 2 μM), Me-CpG-ODN (2006 methylated, 2 μM; TZGTZGTTTTGTZGTTTTGTZGTT, Z=5-methylcytidine, SEQ ID NO:17), LPS (100 ng/ml) or media, as measured by monitoring NF-κB activation. Similar results were obtained utilizing IL-8 production with the stable clone 293-hTLR9. 293-mTLR9-luc were also stimulated with CpG-ODN (1668, 2μM; TCCATGACGTTCCTGATGCT, SEQ ID NO:18), GpC-ODN (1668-GC, 2 μM; TCCATGAGCTTCCTGATGCT, SEQ ID NO:19), Me-CpG-ODN (1668 methylated, 2 μM; TCCATGAZGTTCCTGATGCT, Z=5-methylcytidine, SEQ ID NO:20), LPS (100 ng/ml) or media, as measured by monitoring NF-κB activation (FIG. 5). Similar results were obtained utilizing IL-8 production with the stable clone 293-mTLR9. Results are representative of at least two independent experiments. These results demonstrate that CpG-DNA non-responsive cell lines can be stably genetically complemented with TLR9 to become responsive to CpG-DNA in a motif-specific manner. These cells can be used for screening of optimal ligands for innate immune responses driven by TLR9 in multiple species.

### Example 11

### Reconstitution of TLR3 Signaling in 293 Fibroblasts

Human TLR3 cDNA and murine TLR3 cDNA in pT-Adv vector (from Clonetech) were individually cloned into the expression vector pcDNA3.1 (−) from Invitrogen using the EcoRI site. The resulting expression vectors mentioned above were transfected into CpG-DNA non-responsive human 293 fibroblast cells (ATCC, CRL-1573) using the calcium phosphate method. Utilizing a “gain of function” assay it was possible to reconstitute human TLR3 (hTLR3) and murine TLR3 (mTLR3) signaling in 293 fibroblast cells.

Since NF-κB activation is central to the IL-1/TLR signal transduction pathway (Medzhitov R et al. (1998) Mol Cell 2:253-8; Muzio M et al. (1998) J Exp Med 187:2097-101), in a first set of experiments human 293 fibroblast cells were transfected with hTLR3 alone or co-transfected with hTLR3 and an NF-κB-driven luciferase reporter construct.

Likewise, in a second set of experiments, 293 fibroblast cells were transfected with hTLR3 alone or co-transfected with hTLR3 and an IFN-α4-driven luciferase reporter construct (described in Example 2 above).

In a third group of experiments, 293 fibroblast cells were transfected with hTLR3 alone or co-transfected with hTLR3 and a RANTES-driven luciferase reporter construct (described in Example 5 above).

### Example 12

### Proline to Histidine Mutation P915H in the TIR Domain of Human and MurineTLR9 Alters TLR9 Signaling

Toll-like receptors have a cytoplasmic Toll/IL-1 receptor (TIR) homology domain which initiates signaling after binding of the adapter molecule MyD88. Medzhitov R et al. (1998) Mol Cell 2:253-8; Kopp E B et al. (1999) Curr Opin Immunol 11:15-8. Reports by others have shown that a single point mutation in the signaling TIR domain in murine TLR4 (Pro712 to His, P712H) or human TLR2 (Pro681 to His, P681H) abolishes host immune response to lipopolysaccharide or gram-positive bacteria, respectively. Poltorak A et al. (1998) Science 282:2085-8; Underhill D M et al. (1999) Nature 401:811-5. Through site-specific mutagenesis the equivalent proline (P) at position 915 of human TLR9 and murine TLR9 were mutated to histidine (H; P915H). These mutations were generated by the use of the primers 5′-GCGACTGGCTGCATGGCAAAACCCTCTTTG-3′ (SEQ ID NO:21) and 5′-CAAAGAGGGTTTTGCCATGCAGCCAGTCGC-3′ (SEQ ID NO:22) for human TLR9 and the primers 5′-CGAGATTGGCTGCATGGCCAGACGCTCTTC-3′ (SEQ ID NO:23) and 5′-GAAGAGCGTCTGGCCATGCAGCCAATCTCG-3′ (SEQ ID NO:24) for murine TLR9. Expression vectors for the mutant TLR9s, hTLR9-P915H and mTLR9-P915H, were constructed and verified using standard recombinant DNA techniques.

For the stimulation of human TLR9 variant, hTLR9-P915H, 293 cells were transiently transfected with expression vector for hTLR9 or hTLR9-P915H and stimulated after 16 hours with ODN 2006 or ODN 1668 at various concentrations. Likewise for the stimulation of murine TLR9 variant, mTLR9-P915H, 293 cells were transiently transfected with expression vector for mTLR9 or mTLR9-P915H and stimulated after 16 hours with ODN 2006 or ODN 1668 at various concentrations. After 48 hours of stimulation, supernatant was harvested and IL-8 production was measured by ELISA. Results demonstrated that TLR9 activity can be destroyed by the P915H mutation in the TIR domain of both human and murine TLR9.

### Example 13

### Exchange of the TIR Domain Between Human TLR3 and Human TLR9 (hTLR3-TIR9 and hTLR9-TIR3)

While TLR3 and TLR9 share many structural features, TLR3, by virtue of its having an alanine rather than proline at a critical position in the TIR domain, may not be able to signal via MyD88 as does TLR9. The chimeric TLRs described here can be used in the screening assays of the invention. To generate molecules consisting of human extracellular TLR3 and the TIR domain of human TLR9 (hTLR3-TIR9), the following approach can be used. Through site-specific mutagenesis a ClaI restriction site is introduced in human TLR3 and human TLR9. For human TLR9 the DNA sequence 5′-GGCCTCAGCATCTTT-3′ (3026-3040, SEQ ID NO:25) is mutated to 5′-GGCCTATCGATTTTT-3′ (SEQ ID NO:26), introducing a ClaI site (underlined in the sequence) but leaving the amino acid sequence (GLSIF, aa 798-802) unchanged. For human TLR3 the DNA sequence 5′-GGGTTCCCAGTGAGA-3′ (2112-2126, SEQ ID NO:27) is mutated to 5′-GGGTTATCGATTAGA-3′ (SEQ ID NO:28), introducing a ClaI site and creating the amino acid sequence (GLSIR, aa 685-689) which differs in three positions (aa 686, 687, 688) from the wildtype human TLR3 sequence (GFPVR, aa 685-689).

hTLR3-TIR9. The primers used for human TLR9 are 5′-CAGCTCCAGGGCCTATCGATTTTTGCACAGGACC-3′ (SEQ ID NO:29) and 5′-GGTCCTGTGCAAAAATCGATAGGCCCTGGAGCTG-3′ (SEQ ID NO:30). For creating an expression vector containing the extracellular portion of human TLR3 connected to the TIR domain of human TLR9, the human TLR3 expression vector is cut with ClaI and limiting amounts of EcoRI and the fragment coding for the TIR domain of human TLR9 generated by a ClaI and EcoRI digestion of human TLR9 expression vector is ligated in the vector fragment containing the extracellular portion of hTLR3. Transfection into E. coli yields the expression vector hTLR3-TIR9 (human extracellular TLR3-human TLR9 TIR domain). The expressed product of hTLR3-TIR9 can interact with TLR3 ligands and also signal through an MyD88-mediated signal transduction pathway.

hTLR9-TIR3. A fusion construct with the extracellular domain of hTLR9 and the TIR domain of hTLR3 is prepared using an analogous strategy. For creating an expression vector containing the extracellular portion of human TLR9 connected to the TIR domain of human TLR3, the human TLR9 expression vector is cut with ClaI and limiting amounts of EcoRI and the fragment coding for the TIR domain of human TLR3 generated by a ClaI and EcoRI digestion of human TLR3 expression vector is ligated in the vector fragment containing the extracellular portion of hTLR9. Transfection into E. coli yields the expression vector hTLR9-TIR3 (human extracellular TLR9-human TLR3 TIR domain). The expressed product of hTLR9-TIR3 can interact with TLR9 ligands, e.g., CpG DNA, and signal through a signal transduction pathway in a manner like TLR3.

### Example 14

### Sensitive in vitro Assay for Detecting Ligand Affinity Differences for a TLR

Human 293 fibroblast cells stably transfected with murine TLR9 and an NF-κB-luciferase reporter were stimulated for 16 hours with the following fully phosphorothioated oligodeoxynucleotides (ODN):

(SEQ ID NO:31)

| | |
|---|---|
| 5890: | T*C*C*A*T*G*A*C*G*T*T*T*T*T*G*A*T*G*T*T |

(SEQ ID NO:32)

| | |
|---|---|
| 5895: | T*C*C*A*T*G*A*C*G*T*T*T*T*T*G*A*T*G |

(SEQ ID NO:33)

| | |
|---|---|
| 5896: | T*C*C*A*T*G*A*C*G*T*T*T*T*T*G*A |

(SEQ ID NO:34)

| | |
|---|---|
| 5897: | T*C*C*A*T*G*A*C*G*T*T*T*T*T |

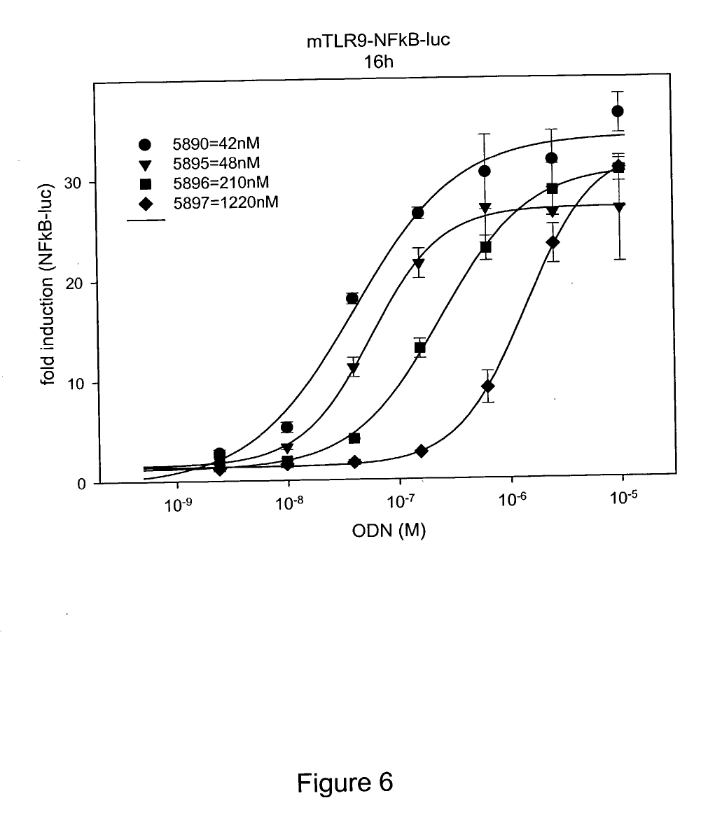

Concentration of the stimulus was titrated between 10 μM and 2 nM. The data is plotted in FIG. 6 as fold induction of NF-κB luciferase, relative to unstimulated background, versus ODN concentration. The data displays typical first-order binding from which EC50 or maximal activity can be determined. EC50 is defined as the concentration of the ligand stimulus that results in 50% maximal activation. As shown in the figure, the EC50 ranges from 42 nM for ODN 5890 to 1220 nM for ODN 5897. The assay demonstrates sensitive differentiation between subtle changes in ligand.

### Example 15

### Influence of Assay Kinetics on TLR Screening Assays

Curves were prepared as in the previous Example 14 with the following ODN ligands, where * indicates phosphrothioate and _ indicates phosophodiester linkage:

| | | |
|---|---|---|
| 5890: | T*C*C*A*T*G*A*C*G*T*T*T*T*T*G*A*T*G*T*T | (SEQ ID NO:35) |
| 5497: | T*C*G*T*C*G*T*T*T*T_G_T_C_G_T*T*T*T*G*T*C*G*T*T | (SEQ ID NO:36) |
| 5746: | T*C_G*T*C_G*T*T*T*T_G*T*C_G*T*T*T*T*G*T*C_G*T*T | (SEQ ID NO:37) |
| 2006: | T*C*G*T*C*G*T*T*T*T*G*T*C*G*T*T*T*T*G*T*C*G*T*T | (SEQ ID NO:15) |
| 5902: | T*C*C*A*T*G*A*C_G_T*T*T*T*T*G*A*T_G*T*T | (SEQ ID NO:38) |

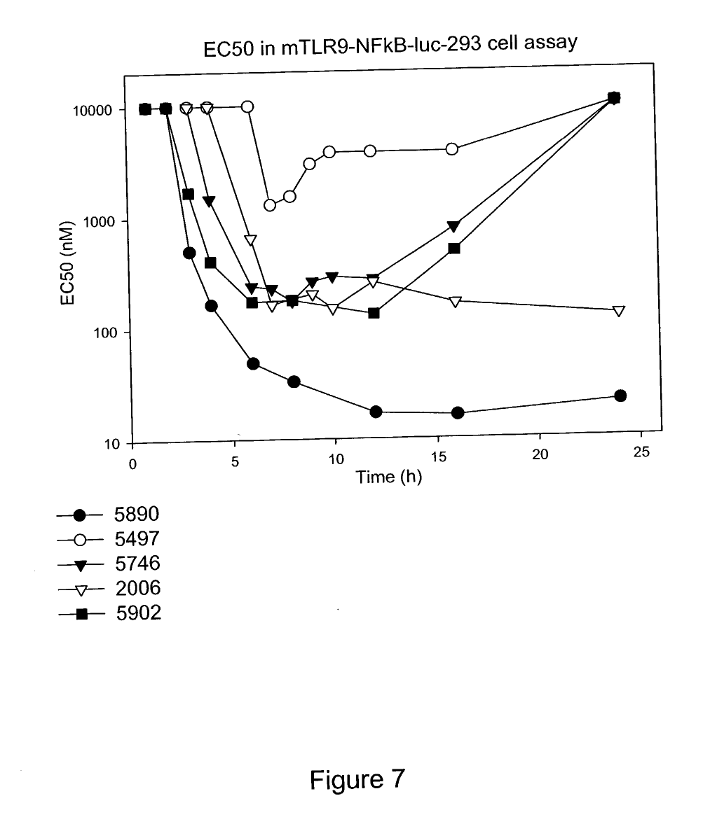

A family of stimulation curves was determined at various times of assay incubation between 1 and 24 hours. The EC50 was determined for each ligand at each time point. The EC50 was then plotted versus time to yield the resultant curves shown in FIG. 7.

As evident from FIG. 7, it is demonstrated that the kinetics of activation vary dependent on the ligand tested. Because luciferase has a three-hour half-life, the signal is transient and requires constant promoter-driven activation to be maintained. The maintenance is directly related to the signal delivered by the ligand/receptor complex. Thus analysis of time kinetics in such a fashion allows one to determine both affinity of ligand/receptor interaction and the availability of the ligand to the receptor through time. The principle is demonstrated as follows. The ODN 5890 is of higher affinity compared to the ODN 2006. When the ligand is made more labile to destruction by incorporating less stable diester linkages, the activity curves turn upward with time such as for ODN 5746, 5902 and 5497.

In the context of a screening assay for TLR/ligand interactions, limiting the assay to one time point would bias the assay. At 24 hours it would appear that only ODN 2006 and 5890 were ligand candidates, however this is clearly not the case. The assay also demonstrates that earlier time points, such as 6 hours in this example, would be the optimal time point for determining the greatest difference between receptor/ligand affinities. Thus optimization of the screening assay can be adjusted depending on the desired information to be obtained from the screen, e.g., higher affinity of interaction versus stability and duration of receptor/ligand interaction.

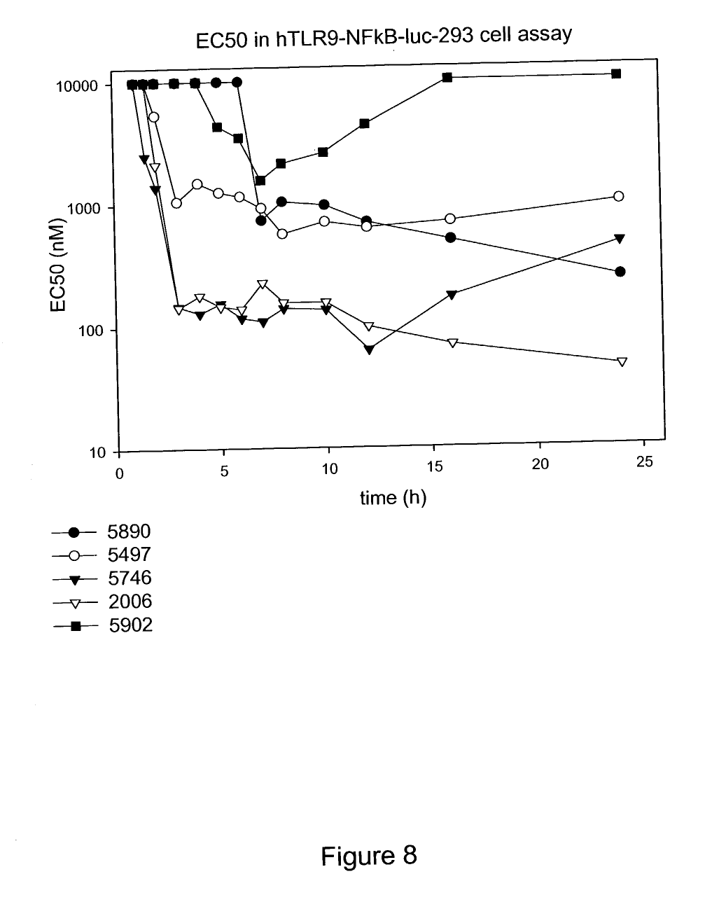

FIG. 8 demonstrates the same principles shown with a murine TLR as in this example can be applied independent of the TLR utilized. For this set of data a 293 cell stably transfected with human TLR9 and NF-κB-luciferase was used.

### Example 16

### Influence of Assay Kinetics on Maximal Activities in TLR Screening Assays

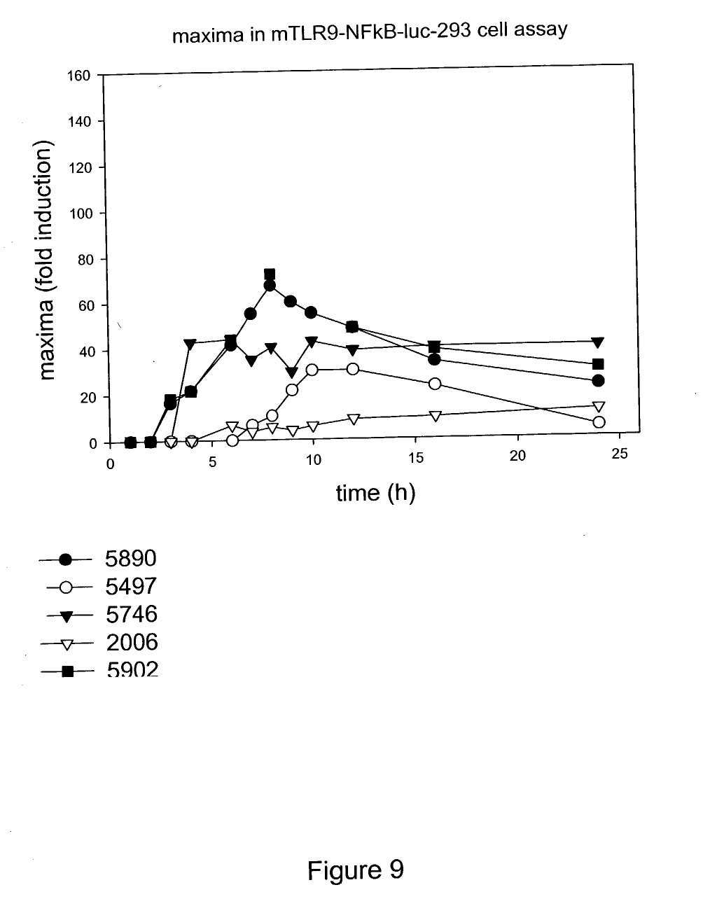

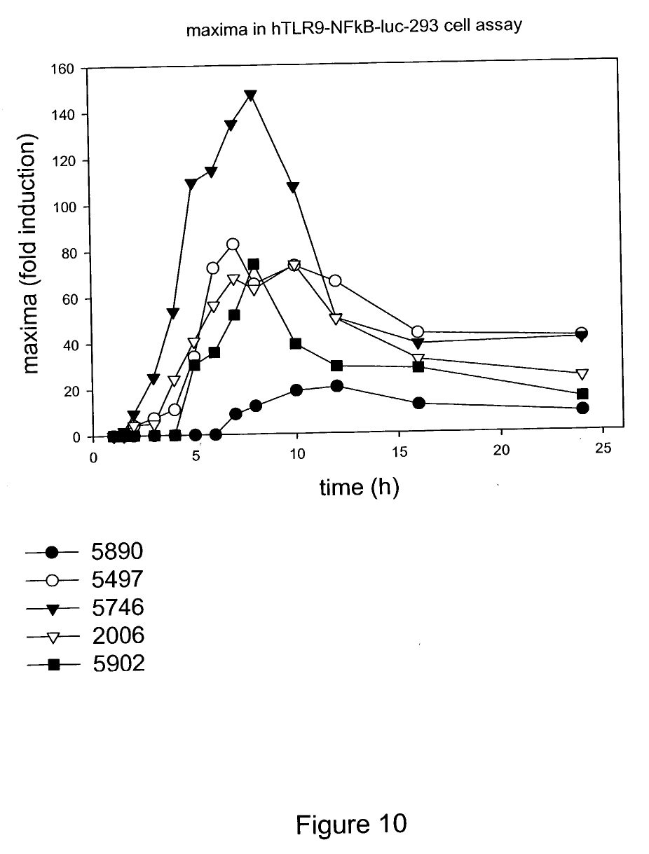

Data was collected as in the previous Example 15, however the maximal activity (maximal fold induction) was plotted versus time in FIGS. 9 and 10. Such data analysis results in a prediction of biological efficacy. As can be seen from these figures, the lower affinity ODN, e.g., ODN 2006 and 5890 as demonstrated by the EC50 curves of Example 15, are clearly less efficient at delivering high activity.

### Example 17

### Differential Outcomes of TLR Screening Assays Dependent on Promoter Utilization

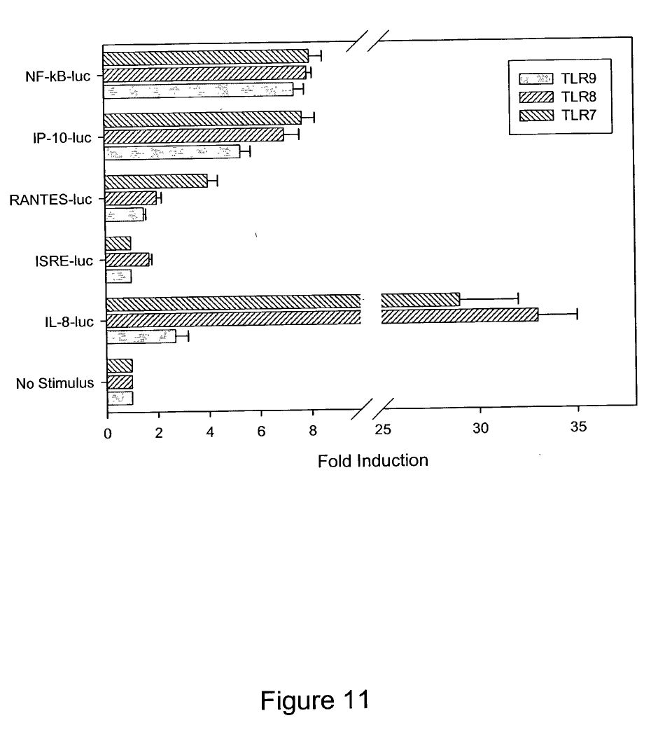

Human 293 fibroblast cells were transiently transfected with expression vector for TLR 7, TLR8, or TLR9 and one of the following reporter constructs bearing the following promoters driving the luciferase gene: NF-κB-luc, IP-10-luc, RANTES-luc, ISRE-luc, and IL-8-luc. The cells were stimulated for 16 h with the maximal activity concentration of specific ligand. TLR9 was stimulated with CpG ODN 2006; TLR8 and TLR7 were stimulated with the imidazolquinalone R848. Results are shown in FIG. 11. As evident from the figure, the promoter used influences the outcome of the screening assay dependent on the TLR in question. For example, NF-κB is a reliable marker for all TLRs tested, whereas in this set of experiments ISRE was only functional to some extent for TLR8. The IL-8 promoter is particularly sensitive for TLR7 or TLR8 screening assays but would be much less efficient in TLR9 assays.

What is claimed is:

1. A screening method for identifying an immunostimulatory compound, comprising:
contacting a functional TLR3 with a test compound under conditions which, in absence of the test compound, permit a negative control response mediated by a TLR3 signal transduction pathway;
detecting a test response mediated by the TLR3 signal transduction pathway; and
determining the test compound is an immunostimulatory compound when the test response exceeds the negative control response.

2. A screening method for identifying an immunostimulatory compound, comprising:
contacting a functional TLR3 with a test compound under conditions which, in presence of a reference immunostimulatory compound, permit a reference response mediated by a TLR3 signal transduction pathway;
detecting a test response mediated by the TLR3 signal transduction pathway; and
determining the test compound is an immunostimulatory compound when the test response equals or exceeds the reference response.

3. A screening method for identifying a compound that modulates TLR3 signaling activity, comprising:
contacting a functional TLR3 with a test compound and a reference immunostimulatory compound under conditions which, in presence of the reference immunostimulatory compound alone, permit a reference response mediated by a TLR3 signal transduction pathway;
detecting a test-reference response mediated by the TLR3 signal transduction pathway;
determining the test compound is an agonist of TLR3 signaling activity when the test-reference response exceeds the reference response; and
determining the test compound is an antagonist of TLR3 signaling activity when the reference response exceeds the test-reference response.

4. A screening method for identifying species specificity of an immunostimulatory compound, comprising:
measuring a first species-specific response mediated by a TLR3 signal transduction pathway when a functional TLR3 of a first species is contacted with a test compound;
measuring a second species-specific response mediated by the TLR3 signal transduction pathway when a functional TLR3 of a second species is contacted with the test compound; and
comparing the first species-specific response with the second species-specific response.

5. The method of any one of claims 1-4, wherein the screening method is performed on a plurality of test compounds.

6. The method of claim 5, wherein the response mediated by the TLR3 signal transduction pathway is measured quantitatively.

7. The method of any one of claims 1-4, wherein the functional TLR3 is expressed in a cell.

8. The method of claim 7, wherein the cell is an isolated mammalian cell that naturally expresses the functional TLR3.

9. The method of claim 7, wherein the cell is an isolated mammalian cell that does not naturally express the functional TLR3, and wherein the cell comprises an expression vector for TLR3.

10. The method of claim 9, wherein the cell is a 293 human fibroblast.

11. The method of claim 7, wherein the cell comprises an expression vector comprising an isolated nucleic acid which encodes a reporter construct selected from the group of interleukin-6-luciferase (IL-6-luc), IL-8-luc, IL-12 p40-luc, IL-12 p40-β-Gal, NF-κB-luc, API-luc, IFN-α-luc, IFN-β-luc, RANTES-luc, TNF-luc, IP-10-luc, I-TAC-luc, and ISRE-luc.

12. The method of claim 11, wherein the reporter construct is ISRE-luc.

13. The method of any one of claims 1-4, wherein the functional TLR3 is part of a cell-free system.

14. The method of any one of claims 1-4, wherein the functional TLR3 is part of a complex with a non-TLR protein selected from the group consisting of MyD88, IL-1 receptor associated kinase 1-3 (IRAK1, IRAK2, IRAK3), tumor necrosis factor receptor-associated factor 1-6 (TRAF1-TRAF6), IκB, NF-κB, MyD88-adapter-like (Mal), Toll-interleukin 1 receptor (TIR) domain-containing adapter protein (TIRAP), Tollip, Rac, and functional homologues and derivatives thereof.

15. The method of claim 14, wherein the non-TLR protein excludes MyD88.

16. The method of claim 2 or 3, wherein the reference immunostimulatory compound is a nucleic acid.

17. The method of claim 16, wherein the nucleic acid is a CpG nucleic acid.

18. The method of claim 2 or 3, wherein the reference immunostimulatory compound is a small molecule.

19. The method of any one of claims 1-4, wherein the test compound is a part of a combinatorial library of compounds.

20. The method of any one of claims 1-4, wherein the test compound is a nucleic acid.

21. The method of claim 20, wherein the nucleic acid is a CpG nucleic acid.

22. The method of any one of claims 1-4, wherein the test compound is a small molecule.

23. The method of any one of claims 1-4, wherein the test compound is a polypeptide.

24. The method of any one of claims 1-4, wherein the response mediated by a TLR3 signal transduction pathway is induction of a reporter gene under control of a promoter response element selected from the group consisting of ISRE, IL-6, IL-8, IL-12 p40, IFN-α, IFN-β, IFN-ω, RANTES, TNF, IP-10, and I-TAC.

25. The method of claim 24, wherein the reporter gene under control of a promoter response element is selected from the group consisting of ISRE-luc, IL-6-luc, IL-8-luc, IL-12 p40-luc, IL-12 p40-β-Gal, IFN-α-luc, IFN-β-luc, RANTES-luc, TNF-luc, IP-10-luc, and I-TAC-luc.

26. The method of claim 25, wherein the reporter gene under control of a promoter response element is ISRE-luc.

27. The method of claim 24, wherein the reporter gene is selected from the group consisting of IFN-α1-luc and IFN-α4-luc.

28. The method of any one of claims 1-4, wherein the response mediated by a TLR3 signal transduction pathway is selected from the group consisting of (a) induction of a reporter gene under control of a minimal promoter responsive to a transcription factor selected from the group consisting of AP1, NF-κB, ATF2, IRF3, and IRF7; (b) secretion of a chemokine; and (c) secretion of a cytokine.

29. The method of claim 28, wherein the response mediated by a TLR3 signal transduction pathway is induction of a reporter gene selected from the group consisting of AP1-luc and NF-κB-luc.

30. The method of claim 28, wherein the response mediated by a TLR3 signal transduction pathway is secretion of a type 1 IFN.

31. The method of claim 28, wherein the response mediated by a TLR3 signal transduction pathway is secretion of a chemokine selected from the group consisting of CCL5 (RANTES), CXCL9 (Mig), CXCL10 (IP-10), and CXCL11 (I-TAC).

32. The method of any one of claims 1-3, wherein the contacting a functional TLR3 with a test compound further comprises, for each test compound, contacting with the test compound at each of a plurality of concentrations.

33. The method of any one of claims 1-3, wherein the detecting is performed 6-12 hours following the contacting.

34. The method of any one of claims 1-3, wherein the detecting is performed 16-24 hours following the contacting.


SEQUENCE LISTING (117 sequences)

SEQ ID NO: 1 - DNA, 3029 bp, Homo sapiens
gcggccgcgtcgacgaaatgtctggatttggactaaagaaaaaaggaaaggctagcagtcatccaacagaatcatgagacagactttgccttgtatctacttttgggggggccttttgccctttgggatgctgtgtgcatcctccaccaccaagtgcactgttagccatgaagttgctgactgcagccacctgaagttgactcaggtacccgatgatctacccacaaacataacagtgttgaaccttacccataatcaactcagaagattaccagccgccaacttcacaaggtatagccagctaactagcttggatgtaggatttaacaccatctcaaaactggagccagaattgtgccagaaacttcccatgttaaaagttttgaacctccagcacaatgagctatctcaactttctgataaaacctttgccttctgcacgaatttgactgaactccatctcatgtccaactcaatccagaaaattaaaaataatccctttgtcaagcagaagaatttaatcacattagatctgtctcataatggcttgtcatctacaaaattaggaactcaggttcagctggaaaatctccaagagcttctattatcaaacaataaaattcaagcgctaaaaagtgaagaactggatatctttgccaattcatctttaaaaaaattagagttgtcatcgaatcaaattaaagagttttctccagggtgttttcacgcaattggaagattatttggcctctttctgaacaatgtccagctgggtcccagccttacagagaagctatgtttggaattagcaaacacaagcattcggaatctgtctctgagtaacagccagctgtccaccaccagcaatacaactttcttgggactaaagtggacaaatctcactatgctcgatctttcctacaacaacttaaatgtggttggtaacgattcctttgcttggcttccacaactagaatatttcttcctagagtataataatatacagcatttgttttctcactctttgcacgggcttttcaatgtgaggtacctgaatttgaaacggtcttttactaaacaaagtatttcccttgcctcactccccaagattgatgatttttcttttcagtggctaaaatgtttggagcaccttaacatggaagataatgatattccaggcataaaaagcaatatgttcacaggattgataaacctgaaatacttaagtctatccaactcctttacaagtttgcgaactttgacaaatgaaacatttgtatcacttgctcattctcccttacacatactcaacctaaccaagaataaaatctcaaaaatagagagtgatgctttctcttggttgggccacctagaagtacttgacctgggccttaatgaaattgggcaagaactcacaggccaggaatggagaggtctagaaaatattttcgaaatctatctttcctacaacaagtacctgcagctgactaggaactcctttgccttggtcccaagccttcaacgactgatgctccgaagggtggcccttaaaaatgtggatagctctccttcaccattccagcctcttcgtaacttgaccattctggatctaagcaacaacaacatagccaacataaatgatgacatgttggagggtcttgagaaactagaaattctcgatttgcagcataacaacttagcacggctctggaaacacgcaaaccctggtggtcccatttatttcctaaagggtctgtctcacctccacatccttaacttggagtccaacggctttgacgagatcccagttgaggtcttcaaggatttatttgaactaaagatcatcgatttaggattgaataatttaaacacacttccagcatctgtctttaataatcaggtgtctctaaagtcattgaaccttcagaagaatctcataacatccgttgagaagaaggttttcgggccagctttcaggaacctgactgagttagatatgcgctttaatccctttgattgcacgtgtgaaagtattgcctggtttgttaattggattaacgagacccataccaacatccctgagctgtcaagccactacctttgcaacactccacctcactatcatgggttcccagtgagactttttgatacatcatcttgcaaagacagtgccccctttgaactctttttcatgatcaataccagtatcctgttgatttttatctttattgtacttctcatccactttgagggctggaggatatctttttattggaatgtttcagtacatcgagttcttggtttcaaagaaatagacagacagacagaacagtttgaatatgcagcatatataattcatgcctataaagataaggattgggtctgggaacatttctcttcaatggaaaaggaagaccaatctctcaaattttgtctggaagaaagggactttgaggcgggtgtttttgaactagaagcaattgttaacagcatcaaaagaagcagaaaaattatttttgttataacacaccatctattaaaagacccattatgcaaaagattcaaggtacatcatgcagttcaacaagctattgaacaaaatctggattccattatattggttttccttgaggagattccagattataaactgaaccatgcactctgtttgcgaagaggaatgtttaaatctcactgcatcttgaactggccagttcagaaagaacggataggtgcctttcgtcataaattgcaagtagcacttggatccaaaaactctgtacattaaatttatttaaatattcaattagcaaaggagaaactttctcaatttaaaaagttctatggcaaatttaagttttccataaaggtgttataatttgtttattcatatttgtaaatgattatattctatcacaattacatctcttctaggaaaatgtgtctccttatttcaggcctatttttgacaattgacttaattttacccaaaataaaacatataagcacgcaaaaaaaaaaaaaaaaaa

SEQ ID NO: 2 - protein, 904 aa, Homo sapiens
MRQTLPCIYFWGGLLPFGMLCASSTTKCTVSHEVADCSHLKLTQVPDDLPTNITVLNLTHNQLRRLPAANFTRYSQLTSLDVGFNTISKLEPELCQKLPMLKVLNLQHNELSQLSDKTFAFCTNLTELHLMSNSIQKIKNNPFVKQKNLITLDLSHNGLSSTKLGTQVQLENLQELLLSNNKIQALKSEELDIFANSSLKKLELSSNQIKEFSPGCFHAIGRLFGLFLNNVQLGPSLTEKLCLELANTSIRNLSLSNSQLSTTSNTTFLGLKWTNLTMLDLSYNNLNVVGNDSFAWLPQLEYFFLEYNNIQHLFSHSLHGLFNVRYLNLKRSFTKQSISLASLPKIDDFSFQWLKCLEHLNMEDNDIPGIKSNMFTGLINLKYLSLSNSFTSLRTLTNETFVSLAHSPLHILNLTKNKISKIESDAFSWLGHLEVLDLGLNEIGQELTGQEWRGLENIFEIYLSYNKYLQLTRNSFALVPSLQRLMLRRVALKNVDSSPSPFQPLRNLTILDLSNNNIANINDDMLEGLEKLEILDLQHNNLARLWKHANPGGPIYFLKGLSHLHILNLESNGFDEIPVEVFKDLFELKIIDLGLNNLNTLPASVFNNQVSLKSLNLQKNLITSVEKKVFGPAFRNLTELDMRFNPFDCTCESIAWFVNWINETHTNIPELSSHYLCNTPPHYHGFPVRLFDTSSCKDSAPFELFFMINTSILLIFIFIVLLIHFEGWRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVH

SEQ ID NO: 3 - DNA, 3310 bp, Mus musculus
tagaatatgatacagggattgcacccataatctgggctgaatcatgaaagggtgttcctcttatctaatgtactcctttgggggacttttgtccctatggattcttctggtgtcttccacaaaccaatgcactgtgagatacaacgtagctgactgcagccatttgaagctaacacacatacctgatgatcttccctctaacataacagtgttgaatcttactcacaaccaactcagaagattaccacctaccaactttacaagatacagccaacttgctatcttggatgcaggatttaactccatttcaaaactggagccagaactgtgccaaatactccctttgttgaaagtattgaacctgcaacataatgagctctctcagatttctgatcaaacctttgtcttctgcacgaacctgacagaactcgatctaatgtctaactcaatacacaaaattaaaagcaaccctttcaaaaaccagaagaatctaatcaaattagatttgtctcataatggtttatcatctacaaagttgggaacgggggtccaactggagaacctccaagaactgctcttagcaaaaaataaaatccttgcgttgcgaagtgaagaacttgagtttcttggcaattcttctttacgaaagttggacttgtcatcaaatccacttaaagagttctccccggggtgtttccagacaattggcaagttattcgccctcctcttgaacaacgcccaactgaacccccacctcacagagaagctttgctgggaactttcaaacacaagcatccagaatctctctctggctaacaaccagctgctggccaccagcgagagcactttctctgggctgaagtggacaaatctcacccagctcgatctttcctacaacaacctccatgatgtcggcaacggttccttctcctatctcccaagcctgaggtatctgtctctggagtacaacaatatacagcgtctgtcccctcgctctttttatggactctccaacctgaggtacctgagtttgaagcgagcatttactaagcaaagtgtttcacttgcttcacatcccaacattgacgatttttcctttcaatggttaaaatatttggaatatctcaacatggatgacaataatattccaagtaccaaaagcaataccttcacgggattggtgagtctgaagtacctaagtctttccaaaactttcacaagtttgcaaactttaacaaatgaaacatttgtgtcacttgctcattctcccttgctcactctcaacttaacgaaaaatcacatctcaaaaatagcaaatggtactttctcttggttaggccaactcaggatacttgatctcggccttaatgaaattgaacaaaaactcagcggccaggaatggagaggtctgagaaatatatttgagatctacctatcctataacaaatacctccaactgtctaccagttcctttgcattggtccccagccttcaaagactgatgctcaggagggtggcccttaaaaatgtggatatctccccttcacctttccgccctcttcgtaacttgaccattctggacttaagcaacaacaacatagccaacataaatgaggacttgctggagggtcttgagaatctagaaatcctggattttcagcacaataacttagccaggctctggaaacgcgcaaaccccggtggtcccgttaatttcctgaaggggctgtctcacctccacatcttgaatttagagtccaacggcttagatgaaatcccagtcggggttttcaagaacttattcgaactaaagagcatcaatctaggactgaataacttaaacaaacttgaaccattcatttttgatgaccagacatctctaaggtcactgaacctccagaagaacctcataacatctgttgagaaggatgttttcgggccgccttttcaaaacctgaacagtttagatatgcgcttcaatccgttcgactgcacgtgtgaaagtatttcctggtttgttaactggatcaaccagacccacactaatatctttgagctgtccactcactacctctgtaacactccacatcattattatggcttccccctgaagcttttcgatacatcatcctgtaaagacagcgccccctttgaactcctcttcataatcagcaccagtatgctcctggtttttatacttgtggtactgctcattcacatcgagggctggaggatctctttttactggaatgtttcagtgcatcggattcttggtttcaaggaaatagacacacaggctgagcagtttgaatatacagcctacataattcatgcccataaagacagagactgggtctgggaacatttctccccaatggaagaacaagaccaatctctcaaattttgcctagaagaaagggactttgaagcaggcgtccttggacttgaagcaattgttaatagcatcaaaagaagccgaaaaatcattttcgttatcacacaccatttattaaaagaccctctgtgcagaagattcaaggtacatcacgcagttcagcaagctattgagcaaaatctggattcaattatactgatttttctccagaatattccagattataaactaaaccatgcactctgtttgcgaagaggaatgtttaaatctcattgcatcttgaactggccagttcagaaagaacggataaatgcctttcatcataaattgcaagtagcacttggatctcggaattcagcacattaaactcatttgaagatttggagtcggtaaagggatagatccaatttataaaggtccatcatgaatctaagttttacttgaaagttttgtatatttatttatatgtatagatgatgatattacatcacaatccaatctcagttttgaaatatttcggcttatttcattgacatctggtttattcactccaaataaacacatgggcagttaaaaacatcctctattaatagattacccattaattcttgaggtgtatcacagctttaaagggttttaaatatttttatataaataagactgagagttttataaatgtaattttttaaaactcgagtcttactgtgtagctcagaaaggcctggaaattaatatattagagagtcatgtcttgaacttatttatctctgcctccctctgtctccagagtgttgcttttaagggcatgtagcaccacacccagctatgtacgtgtgggattttataatgctcatttttgagacgtttatagaataaaagataattgcttttatggtataaggctacttgaggtaa

SEQ ID NO: 4 - protein, 905 aa, Mus musculus
MKGCSSYLMYSFGGLLSLWILLVSSTNQCTVRYNVADCSHLKLTHIPDDLPSNITVLNLTHNQLRRLPPTNFTRYSQLAILDAGFNSISKLEPELCQILPLLKVLNLQHNELSQISDQTFVFCTNLTELDLMSNSIHKIKSNPFKNQKNLIKLDLSHNGLSSTKLGTGVQLENLQELLLAKNKILALRSEELEFLGNSSLRKLDLSSNPLKEFSPGCFQTIGKLFALLLNNAQLNPHLTEKLCWELSNTSIQNLSLANNQLLATSESTFSGLKWTNLTQLDLSYNNLHDVGNGSFSYLPSLRYLSLEYNNIQRLSPRSFYGLSNLRYLSLKRAFTKQSVSLASHPNIDDFSFQWLKYLEYLNMDDNNIPSTKSNTFTGLVSLKYLSLSKTFTSLQTLTNETFVSLAHSPLLTLNLTKNHISKIANGTFSWLGQLRILDLGLNEIEQKLSGQEWRGLRNIFEIYLSYNKYLQLSTSSFALVPSLQRLMLRRVALKNVDISPSPFRPLRNLTILDLSNNNIANINEDLLEGLENLEILDFQHNNLARLWKRANPGGPVNFLKGLSHLHILNLESNGLDEIPVGVFKNLFELKSINLGLNNLNKLEPFIFDDQTSLRSLNLQKNLITSVEKDVFGPPFQNLNSLDMRFNPFDCTCESISWFVNWINQTHTNIFELSTHYLCNTPHHYYGFPLKLFDTSSCKDSAPFELLFIISTSMLLVFILVVLLIHIEGWRISFYWNVSVHRILGFKEIDTQAEQFEYTAYIIHAHKDRDWVWEHFSPMEEQDQSLKFCLEERDFEAGVLGLEAIVNSIKRSRKIIFVITHHLLKDPLCRRFKVHHAVQQAIEQNLDSIILIFLQNIPDYKLNHALCLRRGMFKSHCILNWPVQKERINAFHHKLQVALGSRNSAH

SEQ ID NO: 5 - DNA, 3352 bp, Homo sapiens
aggctggtataaaaatcttacttcctctattctctgagccgctgctgcccctgtgggaagggacctcgagtgtgaagcatccttccctgtagctgctgtccagtctgcccgccagaccctctggagaagcccctgccccccagcatgggtttctgccgcagcgccctgcacccgctgtctctcctggtgcaggccatcatgctggccatgaccctggccctgggtaccttgcctgccttcctaccctgtgagctccagccccacggcctggtgaactgcaactggctgttcctgaagtctgtgccccacttctccatggcagcaccccgtggcaatgtcaccagcctttccttgtcctccaaccgcatccaccacctccatgattctgactttgcccacctgcccagcctgcggcatctcaacctcaagtggaactgcccgccggttggcctcagccccatgcacttcccctgccacatgaccatcgagcccagcaccttcttggctgtgcccaccctggaagagctaaacctgagctacaacaacatcatgactgtgcctgcgctgcccaaatccctcatatccctgtccctcagccataccaacatcctgatgctagactctgccagcctcgccggcctgcatgccctgcgcttcctattcatggacggcaactgttattacaagaacccctgcaggcaggcactggaggtggccccgggtgccctccttggcctgggcaacctcacccacctgtcactcaagtacaacaacctcactgtggtgccccgcaacctgccttccagcctggagtatctgctgttgtcctacaaccgcatcgtcaaactggcgcctgaggacctggccaatctgaccgccctgcgtgtgctcgatgtgggcggaaattgccgccgctgcgaccacgctcccaacccctgcatggagtgccctcgtcacttcccccagctacatcccgataccttcagccacctgagccgtcttgaaggcctggtgttgaaggacagttctctctcctggctgaatgccagttggttccgtgggctgggaaacctccgagtgctggacctgagtgagaacttcctctacaaatgcatcactaaaaccaaggccttccagggcctaacacagctgcgcaagcttaacctgtccttcaattaccaaaagagggtgtcctttgcccacctgtctctggccccttccttcgggagcctggtcgccctgaaggagctggacatgcacggcatcttcttccgctcactcgatgagaccacgctccggccactggcccgcctgcccatgctccagactctgcgtctgcagatgaacttcatcaaccaggcccagctcggcatcttcagggccttccctggcctgcgctacgtggacctgtcggacaaccgcatcagcggagcttcggagctgacagccaccatgggggaggcagatggaggggagaaggtctggctgcagcctggggaccttgctccggccccagtggacactcccagctctgaagacttcaggcccaactgcagcaccctcaacttcaccttggatctgtcacggaacaacctggtgaccgtgcagccggagatgtttgcccagctctcgcacctgcagtgcctgcgcctgagccacaactgcatctcgcaggcagtcaatggctcccagttcctgccgctgaccggtctgcaggtgctagacctgtcccgcaataagctggacctctaccacgagcactcattcacggagctaccgcgactggaggccctggacctcagctacaacagccagccctttggcatgcagggcgtgggccacaacttcagcttcgtggctcacctgcgcaccctgcgccacctcagcctggcccacaacaacatccacagccaagtgtcccagcagctctgcagtacgtcgctgcgggccctggacttcagcggcaatgcactgggccatatgtgggccgagggagacctctatctgcacttcttccaaggcctgagcggtttgatctggctggacttgtcccagaaccgcctgcacaccctcctgccccaaaccctgcgcaacctccccaagagcctacaggtgctgcgtctccgtgacaattacctggccttctttaagtggtggagcctccacttcctgcccaaactggaagtcctcgacctggcaggaaaccggctgaaggccctgaccaatggcagcctgcctgctggcacccggctccggaggctggatgtcagctgcaacagcatcagcttcgtggcccccggcttcttttccaaggccaaggagctgcgagagctcaaccttagcgccaacgccctcaagacagtggaccactcctggtttgggcccctggcgagtgccctgcaaatactagatgtaagcgccaaccctctgcactgcgcctgtggggcggcctttatggacttcctgctggaggtgcaggctgccgtgcccggtctgcccagccgggtgaagtgtggcagtccgggccagctccagggcctcagcatctttgcacaggacctgcgcctctgcctggatgaggccctctcctgggactgtttcgccctctcgctgctggctgtggctctgggcctgggtgtgcccatgctgcatcacctctgtggctgggacctctggtactgcttccacctgtgcctggcctggcttccctggcgggggcggcaaagtgggcgagatgaggatgccctgccctacgatgccttcgtggtcttcgacaaaacgcagagcgcagtggcagactgggtgtacaacgagcttcgggggcagctggaggagtgccgtgggcgctgggcactccgcctgtgcctggaggaacgcgactggctgcctggcaaaaccctctttgagaacctgtgggcctcggtctatggcagccgcaagacgctgtttgtgctggcccacacggaccgggtcagtggtctcttgcgcgccagcttcctgctggcccagcagcgcctgctggaggaccgcaaggacgtcgtggtgctggtgatcctgagccctgacggccgccgctcccgctacgtgcggctgcgccagcgcctctgccgccagagtgtcctcctctggccccaccagcccagtggtcagcgcagcttctgggcccagctgggcatggccctgaccagggacaaccaccacttctataaccggaacttctgccagggacccacggccgaatagccgtgagccggaatcctgcacggtgccacctccacactcacctcacctctgcctgcctggtctgaccctcccctgctcgcctccctcaccccacacctgacacagagca

SEQ ID NO: 6 - protein, 1032 aa, Homo sapiens [sequence omitted]

SEQ ID NO: 7 - DNA, 3200 bp, Mus musculus
tgtcagagggagcctcgggagaatcctccatctcccaacatggttctccgtcgaaggactctgcaccccttgtccctcctggtacaggctgcagtgctggctgagactctggccctgggtaccctgcctgccttcctaccctgtgagctgaagcctcatggcctggtggactgcaattggctgttcctgaagtctgtaccccgtttctctgcggcagcatcctgctccaacatcacccgcctctccttgatctccaaccgtatccaccacctgcacaactccgacttcgtccacctgtccaacctgcggcagctgaacctcaagtggaactgtccacccactggccttagccccctgcacttctcttgccacatgaccattgagcccagaaccttcctggctatgcgtacactggaggagctgaacctgagctataatggtatcaccactgtgccccgactgcccagctccctggtgaatctgagcctgagccacaccaacatcctggttctagatgctaacagcctcgccggcctatacagcctgcgcgttctcttcatggacgggaactgctactacaagaacccctgcacaggagcggtgaaggtgaccccaggcgccctcctgggcctgagcaatctcacccatctgtctctgaagtataacaacctcacaaaggtgccccgccaactgccccccagcctggagtacctcctggtgtcctataacctcattgtcaagctggggcctgaagacctggccaatctgacctcccttcgagtacttgatgtgggtgggaattgccgtcgctgcgaccatgcccccaatccctgtatagaatgtggccaaaagtccctccacctgcaccctgagaccttccatcacctgagccatctggaaggcctggtgctgaaggacagctctctccatacactgaactcttcctggttccaaggtctggtcaacctctcggtgctggacctaagcgagaactttctctatgaaagcatcaaccacaccaatgcctttcagaacctaacccgcctgcgcaagctcaacctgtccttcaattaccgcaagaaggtatcctttgcccgcctccacctggcaagttccttcaagaacctggtgtcactgcaggagctgaacatgaacggcatcttcttccgctcgctcaacaagtacacgctcagatggctggccgatctgcccaaactccacactctgcatcttcaaatgaacttcatcaaccaggcacagctcagcatctttggtaccttccgagcccttcgctttgtggacttgtcagacaatcgcatcagtgggccttcaacgctgtcagaagccacccctgaagaggcagatgatgcagagcaggaggagctgttgtctgcggatcctcacccagctccactgagcacccctgcttctaagaacttcatggacaggtgtaagaacttcaagttcaccatggacctgtctcggaacaacctggtgactatcaagccagagatgtttgtcaatctctcacgcctccagtgtcttagcctgagccacaactccattgcacaggctgtcaatggctctcagttcctgccgctgactaatctgcaggtgctggacctgtcccataacaaactggacttgtaccactggaaatcgttcagtgagctaccacagttgcaggccctggacctgagctacaacagccagccctttagcatgaagggtataggccacaatttcagttttgtggcccatctgtccatgctacacagccttagcctggcacacaatgacattcatacccgtgtgtcctcacatctcaacagcaactcagtgaggtttcttgacttcagcggcaacggtatgggccgcatgtgggatgaggggggcctttatctccatttcttccaaggcctgagtggcctgctgaagctggacctgtctcaaaataacctgcatatcctccggccccagaaccttgacaacctccccaagagcctgaagctgctgagcctccgagacaactacctatctttctttaactggaccagtctgtccttcctgcccaacctggaagtcctagacctggcaggcaaccagctaaaggccctgaccaatggcaccctgcctaatggcaccctcctccagaaactggatgtcagcagcaacagtatcgtctctgtggtcccagccttcttcgctctggcggtcgagctgaaagaggtcaacctcagccacaacattctcaagacggtggatcgctcctggtttgggcccattgtgatgaacctgacagttctagacgtgagaagcaaccctctgcactgtgcctgtggggcagccttcgtagacttactgttggaggtgcagaccaaggtgcctggcctggctaatggtgtgaagtgtggcagccccggccagctgcagggccgtagcatcttcgcacaggacctgcggctgtgcctggatgaggtcctctcttgggactgctttggcctttcactcttggctgtggccgtgggcatggtggtgcctatactgcaccatctctgcggctgggacgtctggtactgttttcatctgtgcctggcatggctacctttgctggcccgcagccgacgcagcgcccaagctctcccctatgatgccttcgtggtgttcgataaggcacagagcgcagttgcggactgggtgtataacgagctgcgggtgcggctggaggagcggcgcggtcgccgagccctacgcttgtgtctggaggaccgagattggctgcctggccagacgctcttcgagaacctctgggcttccatctatgggagccgcaagactctatttgtgctggcccacacggaccgcgtcagtggcctcctgcgcaccagcttcctgctggctcagcagcgcctgttggaagaccgcaaggacgtggtggtgttggtgatcctgcgtccggatgcccaccgctcccgctatgtgcgactgcgccagcgtctctgccgccagagtgtgctcttctggccccagcagcccaacgggcaggggggcttctgggcccagctgagtacagccctgactagggacaaccgccacttctataaccagaacttctgccggggacctacagcagaatagctcagagcaacagctggaaacagctgcatcttcatgcctggttcccgagttgctctgcctgc

SEQ ID NO: 8 - protein, 1032 aa, Mus musculus
MVLRRRTLHPLSLLVQAAVLAETLALGTLPAFLPCELKPHGLVDCNWLFLKSVPRFSAAASCSNITRLSLISNRIHHLHNSDFVHLSNLRQLNLKWNCPPTGLSPLHFSCHMTIEPRTFLAMRTLEELNLSYNGITTVPRLPSSLVNLSLSHTNILVLDANSLAGLYSLRVLFMDGNCYYKNPCTGAVKVTPGALLGLSNLTHLSLKYNNLTKVPRQLPPSLEYLLVSYNLIVKLGPEDLANLTSLRVLDVGGNCRRCDHAPNPCIECGQKSLHLHPETFHHLSHLEGLVLKDSSLHTLNSSWFQGLVNLSVLDLSENFLYESINHTNAFQNLTRLRKLNLSFNYRKKVSFARLHLASSFKNLVSLQELNMNGIFFRSLNKYTLRWLADLPKLHTLHLQMNFINQAQLSIFGTFRALRFVDLSDNRISGPSTLSEATPEEADDAEQEELLSADPHPAPLSTPASKNFMDRCKNFKFTMDLSRNNLVTIKPEMFVNLSRLQCLSLSHNSIAQAVNGSQFLPLTNLQVLDLSHNKLDLYHWKSFSELPQLQALDLSYNSQPFSMKGIGHNFSFVAHLSMLHSLSLAHNDIHTRVSSHLNSNSVRFLDFSGNGMGRMWDEGGLYLHFFQGLSGLLKLDLSQNNLHILRPQNLDNLPKSLKLLSLRDNYLSFFNWTSLSFLPNLEVLDLAGNQLKALTNGTLPNGTLLQKLDVSSNSIVSVVPAFFALAVELKEVNLSHNILKTVDRSWFGPIVMNLTVLDVRSNPLHCACGAAFVDLLLEVQTKVPGLANGVKCGSPGQLQGRSIFAQDLRLCLDEVLSWDCFGLSLLAVAVGMVVPILHHLCGWDVWYCFHLCLAWLPLLARSRRSAQALPYDAFVVFDKAQSAVADWVYNELRVRLEERRGRRALRLCLEDRDWLPGQTLFENLWASIYGSRKTLFVLAHTDRVSGLLRTSFLLAQQRLLEDRKDVVVLVILRPDAHRSRYVRLRQRLCRQSVLFWPQQPNGQGGFWAQLSTALTRDNRHFYNQNFCRGPTAE

SEQ ID NO: 9 - DNA, 42 bp, Artificial sequence
  Synthetic oligonucleotide
gaaactcgagccaccatgagacagactttgccttgtatctac

SEQ ID NO: 10 - DNA, 37 bp, Artificial sequence
  Synthetic oligonucleotide
gaaagaattcttaatgtacagagtttttggatccaag

SEQ ID NO: 11 - DNA, 670 bp, Homo sapiens
agaaaaattttaaaaaattattcattcatatttttaggagttttgaatgattggatatgtaattatattcatattattaatgtgtatctatatagatttttattttgcatatgtactttgatacaaaatttacatgaacaaattacactaaaagttattccacaaatatacttatcaaattaagttaaatgtcaatagcttttaaacttaaattttagtttaacttttctgtcattctttactttgaataaaaagagcaaactttgtagtttttatctgtgaagtagaggtatacgtaatatacataaatagatatgccaaatctgtgttattaaaatttcatgaagatttcaattagaaaaaaataccataaaaggctttgagtgcaggtgaaaaataggcaatgatgaaaaaaaatgaaaaactttttaaacacatgtagagagtgcgtaaagaaagcaaaaacagagatagaaagtacaactagggaatttagaaaatggaaattagtatgttcactatttaagacctatgcacagagcaaagtcttcagaaaacctagaggccgaagttcaaggttatccatctcaagtagcctagcaatatttgcaacatcccaatggccctgtccttttctttactgatggccgtgctggtgctcagctacaaa

SEQ ID NO: 12 - DNA, 300 bp, Homo sapiens
ttctcaggtcgtttgctttcctttgctttctcccaagtcttgttttacaatttgctttagtcattcactgaaactttaaaaaacattagaaaacctcacagtttgtaaatctttttccctattatatatatcataagataggagcttaaataaagagttttagaaactactaaaatgtaaatgacataggaaaactgaaagggagaagtgaaagtgggaaattcctctgaatagagagaggaccatctcatataaataggccatacccacggagaaaggacattctaactgcaacctttc

SEQ ID NO: 13 - DNA, 1031 bp, Homo sapiens
agaaggccttacagtgagatgggatcccagtatttattgagtttcctcattcataaaatggggataataatagtaaatgagttgacacgcgctaagacagtggaatagtggctggcacagataagccctcggtaaatggtagccaataatgatagagtatgctgtaagatatctttctctccctctgcttctcaacaagtctctaatcaattattccactttataaacaaggaaatagaactcaaagacattaagcacttttcccaaaggtcgcttagcaagtaaatgggagagaccctatgaccaggatgaaagcaagaaattcccacaagaggactcattccaactcatatcttgtgaaaaggttcccaatgcccagctcagatcaactgcctcaatttacagtgtgagtgtgctcacctcctttggggactgtatatccagaggaccctcctcaataaaacactttataaataacatccttccatggatgagggaaaggaggtaagatctgtaatgaataagcaggaactttgaagactcagtgactcagtgagtaataaagactcagtgacttctgatcctgtcctaactgccactccttgttgtccccaagaaagcggcttcctgctctctgaggaggaccccttccctggaaggtaaaactaaggatgtcagcagagaaatttttccaccattggtgcttggtcaaagaggaaactgatgagctcactctagatgagagagcagtgagggagagacagagactcgaatttccggaggctatttcagttttcttttccgttttgtgcaatttcacttatgataccggccaatgcttggttgctattttggaaactccccttaggggatgcccctcaactggccctataaagggccagcctgagctgcagaggattcctgcagaggatcaagacagcacgtggacctcgcacagcctctcccacaggtaccatgaaggtctccgcggcagccctcgctgtcatcctcattgctactgccctctgcgc

SEQ ID NO: 14 - DNA, 401 bp, Homo sapiens
gatctgtaatgaataagcaggaactttgaagactcagtgactcagtgagtaataaagactcagtgacttctgatcctgtcctaactgccactccttgttgtcccaagaaagcggcttcctgctctctgaggaggaccccttccctggaaggtaaaactaaggatgtcagcagagaaatttttccaccattggtgcttggtcaaagaggaaactgatgagctcactctagatgagagagcagtgagggagagacagagactcgaatttccggagctatttcagttttcttttccgttttgtgcaatttcacttatgataccggccaatgcttggttgctattttggaaactccccttaggggatgcccctcaactggccctataaagggccagcctgagctg

SEQ ID NO: 15 - DNA, 24 bp, Artificial sequence
  Synthetic oligonucleotide
tcgtcgttttgtcgttttgtcgtt

SEQ ID NO: 16 - DNA, 24 bp, Artificial sequence
  Synthetic oligonucleotide
tgctgcttttgtgcttttgtgctt

SEQ ID NO: 17 - DNA, 24 bp, Artificial sequence
  Synthetic oligonucleotide
tngtngttttgtngttttgtngtt

SEQ ID NO: 18 - DNA, 20 bp, Artificial sequence
  Synthetic oligonucleotide
tccatgacgttcctgatgct

SEQ ID NO: 19 - DNA, 20 bp, Artificial sequence
  Synthetic oligonucleotide
tccatgagcttcctgatgct

SEQ ID NO: 20 - DNA, 20 bp, Artificial sequence
  Synthetic oligonucleotide
tccatgangttcctgatgct

SEQ ID NO: 21 - DNA, 30 bp, Artificial sequence
  Synthetic oligonucleotide
gcgactggctgcatggcaaaaccctctttg

SEQ ID NO: 22 - DNA, 30 bp, Artificial sequence
  Synthetic oligonucleotide
caaagagggttttgccatgcagccagtcgc

SEQ ID NO: 23 - DNA, 30 bp, Artificial sequence
  Synthetic oligonucleotide
cgagattggctgcatggccagacgctcttc

SEQ ID NO: 24 - DNA, 30 bp, Artificial sequence
  Synthetic oligonucleotide
gaagagcgtctggccatgcagccaatctcg

SEQ ID NO: 25 - DNA, 15 bp, Artificial sequence
  Synthetic oligonucleotide
ggcctcagcatcttt

SEQ ID NO: 26 - DNA, 15 bp, Artificial sequence
  Synthetic oligonucleotide
ggcctatcgattttt

SEQ ID NO: 27 - DNA, 15 bp, Artificial sequence
  Synthetic oligonucleotide
gggttcccagtgaga

SEQ ID NO: 28 - DNA, 15 bp, Artificial sequence
  Synthetic oligonucleotide
gggttatcgattaga

SEQ ID NO: 29 - DNA, 34 bp, Artificial sequence
  Synthetic oligonucleotide
cagctccagggcctatcgatttttgcacaggacc

SEQ ID NO: 30 - DNA, 34 bp, Artificial sequence
  Synthetic oligonucleotide
ggtcctgtgcaaaaatcgataggccctggagctg

SEQ ID NO: 31 - DNA, 20 bp, Artificial sequence
  Synthetic oligonucleotide
tccatgacgtttttgatgtt

SEQ ID NO: 32 - DNA, 18 bp, Artificial sequence
  Synthetic oligonucleotide
tccatgacgtttttgatg

SEQ ID NO: 33 - DNA, 16 bp, Artificial sequence
  Synthetic oligonucleotide
tccatgacgtttttga

SEQ ID NO: 34 - DNA, 14 bp, Artificial sequence
  Synthetic oligonucleotide
tccatgacgttttt

SEQ ID NO: 35 - DNA, 20 bp, Artificial sequence
  Synthetic oligonucleotide
tccatgacgtttttgatgtt

SEQ ID NO: 36 - DNA, 24 bp, Artificial sequence
  Synthetic oligonucleotide
tcgtcgttttgtcgttttgtcgtt

SEQ ID NO: 37 - DNA, 24 bp, Artificial sequence
  Synthetic oligonucleotide
tcgtcgttttgtcgttttgtcgtt

SEQ ID NO: 38 - DNA, 20 bp, Artificial sequence
  Synthetic oligonucleotide
tccatgacgtttttgatgtt

SEQ ID NO: 39 - DNA, 20 bp, Artificial sequence
  Synthetic oligonucleotide
aagcgaaaatgaaattgact

SEQ ID NO: 40 - DNA, 24 bp, Artificial sequence
  Synthetic oligonucleotide
accatggacgaactgtttcccctc

SEQ ID NO: 41 - DNA, 24 bp, Artificial sequence
  Synthetic oligonucleotide
accatggacgacctgtttcccctc

SEQ ID NO: 42 - DNA, 24 bp, Artificial sequence
  Synthetic oligonucleotide
accatggacgagctgtttcccctc

SEQ ID NO: 43 - DNA, 24 bp, Artificial sequence
  Synthetic oligonucleotide
accatggacgatctgtttcccctc

SEQ ID NO: 44 - DNA, 24 bp, Artificial sequence
  Synthetic oligonucleotide
accatggacggtctgtttcccctc

SEQ ID NO: 45 - DNA, 24 bp, Artificial sequence
  Synthetic oligonucleotide
accatggacgtactgtttcccctc

SEQ ID NO: 46 - DNA, 24 bp, Artificial sequence
  Synthetic oligonucleotide
accatggacgttctgtttcccctc

SEQ ID NO: 47 - DNA, 20 bp, Artificial sequence
  Synthetic oligonucleotide
agcgggggcgagcgggggcg

SEQ ID NO: 48 - DNA, 18 bp, Artificial sequence
  Synthetic oligonucleotide
agctatgacgttccaagg

SEQ ID NO: 49 - DNA, 20 bp, Artificial sequence
  Synthetic oligonucleotide
atcgactctcgagcgttctc

SEQ ID NO: 50 - DNA, 17 bp, Artificial sequence
  Synthetic oligonucleotide
atgacgttcctgacgtt

SEQ ID NO: 51 - DNA, 20 bp, Artificial sequence
  Synthetic oligonucleotide
atggaaggtccaacgttctc

SEQ ID NO: 52 - DNA, 20 bp, Artificial sequence
  Synthetic oligonucleotide
atggaaggtccagcgttctc

SEQ ID NO: 53 - DNA, 20 bp, Artificial sequence
  Synthetic oligonucleotide
atggactctccagcgttctc

SEQ ID NO: 54 - DNA, 20 bp, Artificial sequence
  Synthetic oligonucleotide
atggaggctccatcgttctc

SEQ ID NO: 55 - DNA, 15 bp, Artificial sequence
  Synthetic oligonucleotide
cacgttgaggggcat

SEQ ID NO: 56 - DNA, 20 bp, Artificial sequence
  Synthetic oligonucleotide
caggcataacggttccgtag

SEQ ID NO: 57 - DNA, 20 bp, Artificial sequence
  Synthetic oligonucleotide
ctgatttccccgaaatgatg

SEQ ID NO: 58 - DNA, 20 bp, Artificial sequence
  Synthetic oligonucleotide
gagaacgatggaccttccat

SEQ ID NO: 59 - DNA, 20 bp, Artificial sequence
  Synthetic oligonucleotide
gagaacgctccagcactgat

SEQ ID NO: 60 - DNA, 20 bp, Artificial sequence
  Synthetic oligonucleotide
gagaacgctcgaccttccat

SEQ ID NO: 61 - DNA, 20 bp, Artificial sequence
  Synthetic oligonucleotide
gagaacgctcgaccttcgat

SEQ ID NO: 62 - DNA, 20 bp, Artificial sequence
  Synthetic oligonucleotide
gagaacgctggaccttccat

SEQ ID NO: 63 - DNA, 20 bp, Artificial sequence
  Synthetic oligonucleotide
gattgcctgacgtcagagag

SEQ ID NO: 64 - DNA, 15 bp, Artificial sequence
  Synthetic oligonucleotide
gcatgacgttgagct

SEQ ID NO: 65 - DNA, 20 bp, Artificial sequence
  Synthetic oligonucleotide
gcggcgggcggcgcgcgccc

SEQ ID NO: 66 - DNA, 21 bp, Artificial sequence
  Synthetic oligonucleotide
gcgtgcgttgtcgttgtcgtt

SEQ ID NO: 67 - DNA, 15 bp, Artificial sequence
  Synthetic oligonucleotide
gctagacgttagcgt

SEQ ID NO: 68 - DNA, 15 bp, Artificial sequence
  Synthetic oligonucleotide
gctagacgttagtgt

SEQ ID NO: 69 - DNA, 15 bp, Artificial sequence
  Synthetic oligonucleotide
gctagatgttagcgt

SEQ ID NO: 70 - DNA, 20 bp, Artificial sequence
  Synthetic oligonucleotide
gcttgatgactcagccggaa

SEQ ID NO: 71 - DNA, 18 bp, Artificial sequence
  Synthetic oligonucleotide
ggaatgacgttccctgtg

SEQ ID NO: 72 - DNA, 19 bp, Artificial sequence
  Synthetic oligonucleotide
ggggtcaacgttgacgggg

SEQ ID NO: 73 - DNA, 19 bp, Artificial sequence
  Synthetic oligonucleotide
ggggtcagtcttgacgggg

SEQ ID NO: 74 - DNA, 20 bp, Artificial sequence
  Synthetic oligonucleotide
gtccatttcccgtaaatctt

SEQ ID NO: 75 - DNA, 18 bp, Artificial sequence
  Synthetic oligonucleotide
taccgcgtgcgaccctct

SEQ ID NO: 76 - DNA, 12 bp, Artificial sequence
  Synthetic oligonucleotide
tcagcgtgcgcc

SEQ ID NO: 77 - DNA, 20 bp, Artificial sequence
  Synthetic oligonucleotide
tccacgacgttttcgacgtt

SEQ ID NO: 78 - DNA, 20 bp, Artificial sequence
  Synthetic oligonucleotide
tccataacgttcctgatgct

SEQ ID NO: 79 - DNA, 20 bp, Artificial sequence
  Synthetic oligonucleotide
tccatagcgttcctagcgtt

SEQ ID NO: 80 - DNA, 20 bp, Artificial sequence
  Synthetic oligonucleotide
tccatcacgtgcctgatgct

SEQ ID NO: 81 - DNA, 20 bp, Artificial sequence
  Synthetic oligonucleotide
tccatgacggtcctgatgct

SEQ ID NO: 82 - DNA, 20 bp, Artificial sequence
  Synthetic oligonucleotide
tccatgacgtccctgatgct

SEQ ID NO: 83 - DNA, 20 bp, Artificial sequence
  Synthetic oligonucleotide
tccatgacgtgcctgatgct

SEQ ID NO: 84 - DNA, 20 bp, Artificial sequence
  Synthetic oligonucleotide
tccatgacgttcctgacgtt

SEQ ID NO: 85 - DNA, 20 bp, Artificial sequence
  Synthetic oligonucleotide
tccatgccggtcctgatgct

SEQ ID NO: 86 - DNA, 20 bp, Artificial sequence
  Synthetic oligonucleotide
tccatgcgtgcgtgcgtttt

SEQ ID NO: 87 - DNA, 20 bp, Artificial sequence
  Synthetic oligonucleotide
tccatgcgttgcgttgcgtt

SEQ ID NO: 88 - DNA, 20 bp, Artificial sequence
  Synthetic oligonucleotide
tccatggcggtcctgatgct

SEQ ID NO: 89 - DNA, 20 bp, Artificial sequence
  Synthetic oligonucleotide
tccatgtcgatcctgatgct

SEQ ID NO: 90 - DNA, 20 bp, Artificial sequence
  Synthetic oligonucleotide
tccatgtcgctcctgatgct

SEQ ID NO: 91 - DNA, 20 bp, Artificial sequence
  Synthetic oligonucleotide
tccatgtcggtcctgatgct

SEQ ID NO: 92 - DNA, 20 bp, Artificial sequence
  Synthetic oligonucleotide
tccatgtcggtcctgctgat

SEQ ID NO: 93 - DNA, 20 bp, Artificial sequence
  Synthetic oligonucleotide
tccatgtcgtccctgatgct

SEQ ID NO: 94 - DNA, 20 bp, Artificial sequence
  Synthetic oligonucleotide
tccatgtcgttcctgatgct

SEQ ID NO: 95 - DNA, 20 bp, Artificial sequence
  Synthetic oligonucleotide
tccatgtcgttcctgtcgtt

SEQ ID NO: 96 - DNA, 20 bp, Artificial sequence
  Synthetic oligonucleotide
tccatgtcgtttttgtcgtt

SEQ ID NO: 97 - DNA, 19 bp, Artificial sequence
  Synthetic oligonucleotide
tcctgacgttcctgacgtt

SEQ ID NO: 98 - DNA, 19 bp, Artificial sequence
  Synthetic oligonucleotide
tcctgtcgttcctgtcgtt

SEQ ID NO: 99 - DNA, 20 bp, Artificial sequence
  Synthetic oligonucleotide
tcctgtcgttccttgtcgtt

SEQ ID NO: 100 - DNA, 20 bp, Artificial sequence
  Synthetic oligonucleotide
tcctgtcgttttttgtcgtt

SEQ ID NO: 101 - DNA, 20 bp, Artificial sequence
  Synthetic oligonucleotide
tccttgtcgttcctgtcgtt

SEQ ID NO: 102 - DNA, 20 bp, Artificial sequence
  Synthetic oligonucleotide
tcgatcggggcggggcgagc

SEQ ID NO: 103 - DNA, 21 bp, Artificial sequence
  Synthetic oligonucleotide
tcgtcgctgtctccgcttctt

SEQ ID NO: 104 - DNA, 27 bp, Artificial sequence
  Synthetic oligonucleotide
tcgtcgctgtctccgcttcttcttgcc

SEQ ID NO: 105 - DNA, 21 bp, Artificial sequence
  Synthetic oligonucleotide
tcgtcgctgtctgcccttctt

SEQ ID NO: 106 - DNA, 21 bp, Artificial sequence
  Synthetic oligonucleotide
tcgtcgctgttgtcgtttctt

SEQ ID NO: 107 - DNA, 14 bp, Artificial sequence
  Synthetic oligonucleotide
tcgtcgtcgtcgtt

SEQ ID NO: 108 - DNA, 20 bp, Artificial sequence
  Synthetic oligonucleotide
tcgtcgttgtcgttgtcgtt

SEQ ID NO: 109 - DNA, 22 bp, Artificial sequence
  Synthetic oligonucleotide
tcgtcgttgtcgttttgtcgtt

SEQ ID NO: 110 - DNA, 18 bp, Artificial sequence
  Synthetic oligonucleotide
tctcccagcgcgcgccat

SEQ ID NO: 111 - DNA, 17 bp, Artificial sequence
  Synthetic oligonucleotide
tctcccagcgggcgcat

SEQ ID NO: 112 - DNA, 18 bp, Artificial sequence
  Synthetic oligonucleotide
tctcccagcgtgcgccat

SEQ ID NO: 113 - DNA, 20 bp, Artificial sequence
  Synthetic oligonucleotide
tgcagattgcgcaatctgca

SEQ ID NO: 114 - DNA, 13 bp, Artificial sequence
  Synthetic oligonucleotide
tgtcgttgtcgtt

SEQ ID NO: 115 - DNA, 19 bp, Artificial sequence
  Synthetic oligonucleotide
tgtcgttgtcgttgtcgtt

SEQ ID NO: 116 - DNA, 25 bp, Artificial sequence
  Synthetic oligonucleotide
tgtcgttgtcgttgtcgttgtcgtt

SEQ ID NO: 117 - DNA, 21 bp, Artificial sequence
  Synthetic oligonucleotide
tgtcgtttgtcgtttgtcgtt